(19) United States
(12) Patent Application Publication
(10) Pub. No.: US 2026/0294541 A1
(43) Pub. Date: Oct. 1, 2026

(54) OPTICAL TREATMENT PLAN GENERATION

(71) Applicant: ALCON INC., Fribourg (CH)

(72) Inventors: Wei WEI, Corona, CA (US); Gangjun LIU, Portland, OR (US); Lingfeng YU, Rancho Santa Margarita, CA (US)

(21) Appl. No.: 19/569,808

(22) Filed: Mar. 17, 2026

Related U.S. Application Data
(60) Provisional application No. 63/777,135, filed on Mar. 25, 2025.

(51) Int. Cl. A61B 34/10; A61B 3/00; A61B 3/117; A61B 5/00; A61F 9/008

(52) U.S. Cl. CPC A61B 34/10; A61B 3/0025; A61B 3/1176; A61B 5/0066; A61F 9/008; A61F 2009/00851; A61F 2009/00887

(57) ABSTRACT

The present disclosure relates to operations that include determining, based on a first optical imaging result obtained using an optical imaging system, one or more biomechanical properties of a target tissue corresponding to an eye. The operations may further include determining, based on the first optical imaging result, one or more geometric properties of the target tissue. The operations may further include generating a customized treatment plan for treatment of the target tissue based on the one or more biomechanical properties and the one or more geometric properties.

### CROSS-REFERENCE TO RELATED APPLICATION

This application claims priority to U.S. Provisional Patent Application No. 63/777,135, filed on Mar. 25, 2025, the disclosure of which is incorporated herein by reference in its entirety.

### FIELD

The present disclosure relates to optical treatment plan generation.

### BACKGROUND

Optical imaging (e.g., Optical Coherence Tomography (OCT)) is often used in ophthalmology to obtain images corresponding to the eye. For example, ophthalmology surgical systems may utilize optical imaging to help facilitate the tasks they perform. For instance, cataract surgery may use images obtained using OCT.

### SUMMARY

The present disclosure relates to operations that include determining, based on a first optical imaging result obtained using an optical imaging system, one or more biomechanical properties of a target tissue corresponding to an eye. The operations may further include determining, based on the first optical imaging result, one or more geometric properties of the target tissue. The operations may further include generating a customized treatment plan for treatment of the target tissue based on the one or more biomechanical properties and the one or more geometric properties.

### BRIEF DESCRIPTION OF THE DRAWINGS

The present disclosure relates to systems and methods for generating optical treatment plans, wherein:

FIG. 1 is a block diagram of an example ophthalmic surgical system that may be used with and/or implement one or more embodiments of the present disclosure related to optical treatment plan generation, according to one or more embodiments of the present disclosure;

FIG. 2 illustrates an example process that may be performed to generate a customized treatment plan, according to one or more embodiments of the present disclosure;

FIG. 3 illustrates an example customized treatment plan, according to one or more embodiments of the present disclosure;

FIG. 4 illustrates various example customized treatment plans for various example target tissues, according to one or more embodiments of the present disclosure;

FIG. 5 illustrates an example customized treatment plan for an example target tissue and an updated example customized treatment plan for an altered target tissue, according to one or more embodiments of the present disclosure;

FIG. 6 is a flow diagram illustrating a method of generating a customized treatment plan for a target tissue based on optical images, according to one or more embodiments of the present disclosure; and

FIG. 7 is a block diagram of an example computing system suitable for use in implementing one or more embodiments of the present disclosure.

### DETAILED DESCRIPTION

The human eye functions to provide vision by obtaining light transmitted through a clear outer portion called the cornea and focusing the image by way of a crystalline lens onto the retina at the back of the eye. The quality of the focused image depends on many factors including the size, shape, and length of the eye, and the shape and transparency of the cornea and the lens. When trauma, age, or disease causes the lens to become less transparent, vision deteriorates because of the diminished light transmitted to the retina. This deficiency in the lens of the eye is medically known as a cataract. An accepted treatment for this condition is surgical removal of the cataractous lens.

Cataract surgery may involve using a laser to facilitate removal of a cataractous lens from an eye. For example, in femtosecond laser-assisted cataract surgery (FLACS), the laser may be used in a number of ways. The laser may be used to make one or multiple incisions into the cornea to provide entry points for surgical tools and for insertion of a replacement lens. The laser may also be used to perform a capsulotomy, which is a cut at an anterior portion of the capsular bag. The capsulotomy provides access to the cataractous lens, which is contained within the capsular bag. The cataractous lens may then be fragmented utilizing the laser. Fragmenting the cataractous lens involves making cuts of the cataractous lens with the laser to segment the cataractous lens into multiple pieces. During FLACS, these pieces may then be emulsified or broken-down using ultrasound energy from a phacoemulsification tool, which ultimately allows the segmented pieces to be aspirated out of the eye.

The biomechanical properties of the cataractous lens are not homogenous and may vary significantly. For example, a clouded portion (e.g., a cataractous portion) of the cataractous lens may be stiffer and more opaque than an unclouded portion (e.g., non-cataractous portion) of the cataractous lens. As a result, different portions of the cataractous lens may respond differently to the laser during fragmentation. For example, a stiffer and more opaque portion of the cataractous lens may require a higher laser intensity and/or a longer duration of laser application in order to segment that portion of the cataractous lens than a less stiff, more transparent portion. Additionally, the cutting pattern (e.g., the cutting location, trajectory, and depth) may vary according to the biomechanical properties of the cataractous lens. For example, a different cutting pattern may be utilized in a region of high stiffness than that utilized in a region that is not as stiff. However, the biomechanical properties of the cataractous lens have not typically been utilized to determine cataract treatment. Consequently, traditional cataract removal plans are not tailored to the biomechanical properties of the cataractous lens that is being removed, which results in removal inefficiencies.

The present application relates to creating a customized treatment plan for treating cataracts based on the determination of biomechanical properties of the cataractous lenses. In particular, biomechanical properties of a cataractous lens may be determined from optical images of the eye. For example, the biomechanical properties may be determined from OCT and/or functional extensions of OCT such as Optical Coherence Elastography (OCE) and/or light attenuation analysis.

Optical imaging may be used to generate images and/or representations of different parts of the eye—e.g., the retina, optic nerve, and anterior segment of the eye. In some instances, optical imaging may include OCT. OCT imaging operates on the principle of low-coherence interferometry, utilizing light waves in a manner similar to how ultrasound uses sound waves. A light source, such as a superluminescent diode or a laser, emits a beam that is split into two paths: one directed at the tissue being imaged and the other toward a reference mirror. The light that reflects back from the tissue and the reference mirror is recombined to create an interference pattern, which is analyzed to generate depth information about the tissue. By scanning the light across the tissue, a two-dimensional or three-dimensional image is constructed. As the light penetrates the tissue, it reflects differently based on the varying densities and structures within the tissue. These differences in reflections are captured and processed to form detailed images of the internal microstructures.

In some instances, optical imaging may be used—directly or indirectly—to identify one or more biomechanical properties of the tissue. The biomechanical properties may include elasticity, stiffness (or hardness), viscoelasticity, opacity, transparency, strain, Poisson's Ratio, density, light scattering intensity, and/or light attenuation. The biomechanical properties may directly or indirectly relate to other biomechanical properties such as the grading and type of the target cataracts, including nuclear severity, cortical severity, posterior subcapsular severity, or other biomechanical properties.

In these and other embodiments, the optical imaging may include functional extensions of OCT such as OCE and/or light attenuation analysis that may be used to identify one or more of the biomechanical properties of the tissue. For example, OCE may be utilized to functionally extend OCT beyond structural imaging to provide insight into biomechanical properties such as elasticity, stiffness (or hardness), strain, Poisson's ratio, and/or viscoelasticity. In OCE, a mechanical force (e.g., acoustic waves, air pulses, or direct compression) may be applied to the tissue to induce deformation, micro-motion, displacement, or waves in the tissue. The deformations, micro-motion, displacement, and/or waves in the tissue may impact the light reflected back from the tissue. OCT images may be processed using OCE techniques such as calculation of elastic modulus, speckle decorrelation, or phase decorrelation which may provide biomechanical property information based on the reflected light. Additionally or alternatively, light attenuation analysis may be performed on the OCT images, which utilizes the light attenuation resolved from OCT signals to provide information about biomechanical properties such as opacity, stiffness, transparency, density, elasticity, and/or strain. In the present disclosure, use of the term “OCT imaging” may refer to capturing of OCT images as well as derivation of any applicable property that may be achieved through the use of OCT images. For example, OCT imaging may refer to obtaining 2D images of tissue, 3D images of tissue, identifying geometric properties (e.g., 2D and/or 3D shapes of tissue), and/or identifying one or more biomechanical properties of the tissue (either in general or at specific locations).

In addition, OCT imaging may be utilized to image tissue before surgery to prepare for the surgery, and during surgery to guide placement of surgical equipment. However, traditional approaches do not use the biomechanical properties which may be determined from OCT imaging (e.g., via OCE or light attenuation analysis) as an aid for generating treatment plans. For example, in FLACS, OCT may be utilized to generate an image of the cataractous lens (e.g., to identify geometric properties of the cataractous lens), but because no biomechanical properties of the target tissue are determined from the OCT scan, a generic or “one-size fits all” removal strategy is typically implemented. Furthermore, these generic removal strategies are typically determined from 2D OCT images. Utilizing 2D images to determine a treatment plan limits the efficiency of the treatment plan because the biomechanical properties and geometric properties of the imaged tissue may not be homogenous through the depth of the imaged tissue. Thus, OCT is underutilized in generating treatment plans because the treatment plans are not based on the biomechanical properties of the target tissue which may be determined from OCT images both before and during surgery.

By contrast, one or more embodiments of the present disclosure relate to generating customized treatment plans from optical imaging results (e.g., from OCT imaging) to be implemented during cataract removal surgery (e.g., a customized treatment plan for FLACS). In particular, in some embodiments, one or more biomechanical properties of a cataractous lens may be determined based on an optical imaging result obtained using an optical imaging system. In these and other embodiments, one or more geometric properties of the cataractous lens may be determined based on the optical imaging result. In these and other embodiments, a customized treatment plan for the cataractous lens may be generated based on the one or more biomechanical properties of the cataractous lens and/or the one or more geometric properties of the cataractous lens.

The embodiments of the present disclosure may be utilized with any suitable system, apparatus or device in which treatment plan customization may be beneficial. For example, in some embodiments, an ophthalmic surgical system may be configured to perform optical imaging and may be configured to also perform customized treatment plan generation based on the geometric properties and biomechanical properties of the cataract. Such customization may allow for the ophthalmic surgical system to generate more accurate and precise cataract treatment plans, which may accordingly result in an improvement in surgical operations. Such embodiments may accordingly increase the accuracy and/or precision in the operations performed, which may improve the efficacy, efficiency, and/or safety of such ophthalmic surgical systems.

By way of particular example, OCT imaging may be utilized in conjunction with a FLACS system. Before FLACS, the patient's eye may be imaged using an OCT system to help prepare a treatment plan for the surgery based on the geometric properties of the target tissue. During FLACS, the patient's eye may be imaged using an OCT imaging system, which may guide the placement of the laser during surgery to generate precise incisions and fragment the cataractous lens.

Generating a customized treatment plan based on the biomechanical properties and/or geometric properties of the target tissue may allow for surgical parameters during FLACS to be improved before surgery. For example, instead of a generalized treatment plan, the treatment plan may be customized based on the shape, opacity, and/or stiffness of the cataractous lens to improve the removal of the cataractous lens as compared to more generic treatment plans. Furthermore, the customized treatment plan may be updated during surgery to allow for surgical parameters to be adjusted during surgery based on the biomechanical and geometric properties of the cataractous lens. Thus, the overall timing, efficacy, and safety of FLACS may be improved.

References to a cataractous lens throughout the present disclosure are intended to encompass both the entirety of the cataractous lens as well as the cataract itself. Furthermore, references to a cataract may refer to the pathological condition of clouding within the cataractous lens or may broadly encompass the cataractous lens as a whole. Additionally, references to a clouded lens portion may encompass a portion of the cataractous lens corresponding to the cataract and references to an unclouded lens portion may encompass a portion of the cataractous lens which is not the cataract.

The embodiments of the present disclosure will be explained with reference to the accompanying figures. It is to be understood that the figures are diagrammatic and schematic representations of such example embodiments, and are not limiting, nor are they necessarily drawn to scale. In the figures, features with like numbers indicate like structure and function unless described otherwise. Further, one or more of the figures and accompanying descriptions are given with respect to customized treatment plan generation in the context of cataract surgery. However, such uses are not meant to be limiting such that the customized treatment plan generation described may be used in any number of different contexts and applications where it may be helpful or applicable.

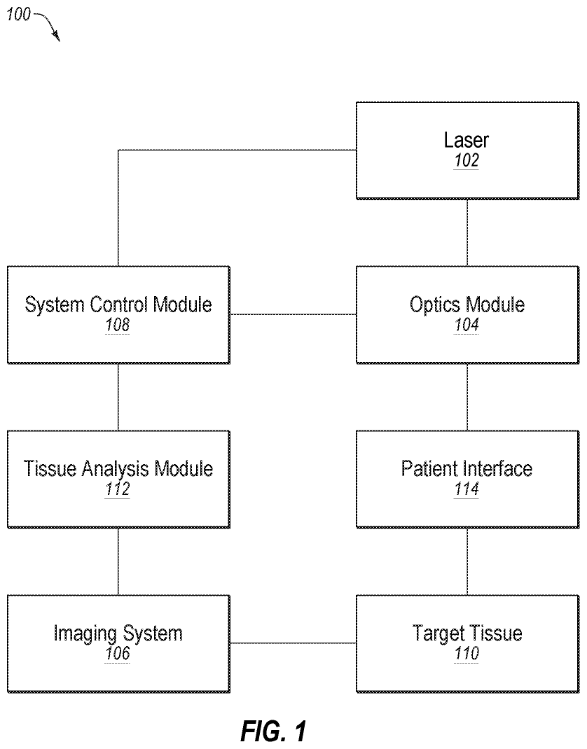

FIG. 1 is a block diagram of an example ophthalmic surgical system 100 (“system 100”) that may be used with and/or implement one or more embodiments of the present disclosure related to optical treatment plan generation. In general, the system 100 may be configured to perform one or more ophthalmic treatment operations with respect to performance of a procedure corresponding to a target tissue 110, which may relate to an eye in some embodiments. For example, in some embodiments, the target tissue 110 may include a cataractous lens and the system 100 may be configured to remove the cataractous lens based on a customized treatment plan that is generated as described in the present disclosure. In some embodiments, the system 100 may include a laser 102, an optics module 104, an imaging system 106, a system control module 108, a target tissue 110, a tissue analysis module 112, and a patient interface 114.

The laser 102 may include any suitable system, apparatus, or device, configured to generate one or more laser beams that may be used to perform ophthalmic operations or tasks with respect to the target tissue 110. In some embodiments, the laser 102 may include multiple lasers that each generate an individual laser beam. Additionally or alternatively, the laser 102 may include a single laser that is configured to generate a single beam or multiple beams.

In some embodiments, the laser 102 may be configured to generate a pulsed laser beam that is pulsed at a high repetition rate at a pulse repetition rate of thousands of shots per second or higher with relatively low energy per pulse. For example, in some embodiments, the laser 102 may be a femtosecond laser that emits ultra-short pulses of light (e.g., on the order of 10\hat{ }-13 seconds). In some embodiments, the laser 102 may emit a laser beam to perform one or more ophthalmic treatment operations. For example, the laser 102 may be used to remove a cataractous lens from an eye. In these and other embodiments, the laser 102 may be operated based on a customized treatment plan for treatment of the target tissue 110. In some embodiments, the properties of the operation of the laser 102 may be adjusted or modified. For example, the system control module 108 may instruct the laser 102 a repetition rate to use at a given instance of time during a treatment procedure, whether or not the laser is to fire, at what wavelength the laser should be firing, at what energy level the laser 102 is to fire, etc.

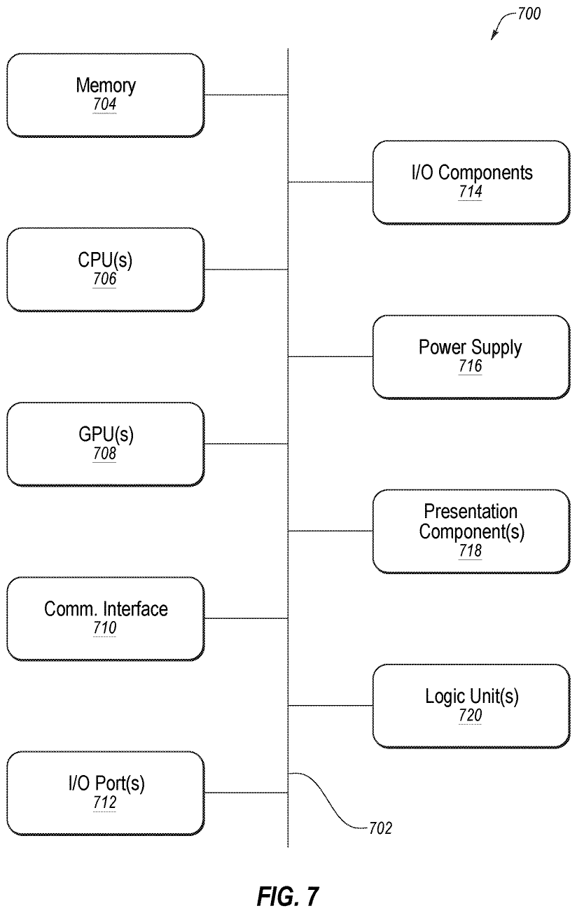

The optics module 104 may include any suitable system, apparatus, or device that may be configured to focus and direct the laser beam to the target tissue 110. For example, in some embodiments, the optics module 104 may include one or more lenses and/or one or more reflectors (e.g., mirrors). Additionally or alternatively, in some embodiments, the optics module 104 may include one or more actuators that may be configured to adjust the focusing and/or the beam direction in response to a beam control signal that may be received from the system control module 108. In these and other embodiments, the one or more actuators may be adjusted in response to a user input via any suitable user interface, such as discussed with respect to the computing system of FIG. 7. For example, in some embodiments, the user interface may include a touch screen, mouse, keyboard, joystick, foot pedal, game pad, game controller, etc., that may be used to provide commands for movement of the laser beam (e.g., via the actuators).

The imaging system 106 may include any suitable system, apparatus, or device, that may be configured to obtain one or more images of the eye corresponding to the target tissue 110. For example, in some embodiments, the imaging system 106 may collect reflected or scattered light or sound from the target tissue 110 to capture image data corresponding to the target tissue 110.

The imaging system 106 may include one or more different types of devices and/or systems configured to capture various different types of images of the eye as corresponding to the target tissue 110. For example, in some embodiments, the imaging system 106 may include a camera configured to capture one or more camera images of the eye. In these and other embodiments, the imaging system may include an ultrasound imaging device configured to capture ultrasound images of the eye.

Additionally or alternatively, the imaging system 106 may include an OCT device configured to capture OCT images of the eye. In these and other embodiments, the OCT images may correspond to various scans of the eye. In these and other embodiments, the OCT device may be configured to perform functional extensions of OCT including OCE and/or light attenuation analysis.

As indicated elsewhere in the present disclosure, OCT imaging may include splitting a beam of light into two different paths, one directed at the target tissue 110 the other toward a reference mirror. The light that reflects back from the tissue and the reference mirror is recombined to create an interference pattern, which may be analyzed to generate information about the target tissue 110. Such information may be used to generate an image of the target tissue 110.

In some embodiments, the imaging system 106 may be configured to perform and/or obtain various OCT scans. For example, the imaging system 106 may perform and/or obtain one or more OCT axial scans (“A-scans”), one or more brightness scans (“B-scans”) that may each be based on multiple A-scans, one or more cross-sectional scans (C-Scans) that may each be based on multiple A-scans, one or more motion/measurement scans (M-scans) that may each be based on multiple A-scans, or other suitable OCT scans.

In some embodiments, the imaging system 106 may be configured to perform multiple OCT axial scans (“A-scans”) to acquire information about the target tissue 110. An OCT A-scan may be a one-dimensional measurement that provides depth information at a single point within the target tissue 110. In particular, the A-scan may measure the time delay and intensity of light as it travels through the tissue and reflects back to a detector of the imaging system 106. The result may be a line graph that represents the reflectivity of different tissue layers along the depth axis. In some embodiments, an A-scan may be used to measure the thickness of various portions of the target tissue 110—such as the lens or other ocular structures—offering precise data about the distance from the surface of the target tissue 110 to various internal layers. In the present disclosure, reference to an “A-scan” may refer to the process of emitting and detecting light with respect to a single point of the target tissue 110, including the depth at that single point, as well as information that may be obtained from such a scan.

In these and other embodiments, the imaging system 106 may be configured to obtain one or more brightness scans (“B-scans”) that may each be based on multiple A-scans. In particular, an OCT B-scan may be a two-dimensional (2D) cross-sectional image of the target tissue 110 that may be constructed from multiple A-scans taken along a line across the target tissue 110. Each A-scan may contribute a vertical line of data, and as the scan progresses across the target tissue 110, these lines may be combined to form a detailed cross-sectional image that forms a corresponding B-scan. This image may show the various layers and structures within portions of the target tissue 110 that are scanned, providing a more comprehensive view of the anatomy. B-scans may be used for diagnosing and monitoring conditions affecting the different portions of the eye such as the grade of cataractous lens, the cornea, the anterior segment, the retina, macula, and optic nerve, as they may reveal abnormalities in tissue structure, thickness, and reflectivity patterns across a broader area. In the present disclosure, reference to a “B-scan” may refer to the process of performing multiple A-scans as well as the cross-sectional image that may be generated from the corresponding A-scans.

In some embodiments, the imaging system 106 may be configured to obtain one or more cross-sectional scans (“C-scans”) that may be based on multiple B-scans. In particular, an OCT C-scan may be a three-dimensional (3D) image of the target tissue 110 that may be constructed from multiple B-scans acquired from different layers of the target tissue 110. This image may show the structural abnormalities and the reflectivity distribution in the target tissue 110. C-scans may be used for diagnosing and monitoring conditions affecting the different portions of the eye as they may reveal abnormalities in tissue structure and reflectivity patterns across a broader area. In the present disclosure, reference to a “C-scan” may refer to the process of performing multiple B-scans as well as the three-dimensional image that may be generated from the corresponding B-scans.

In some embodiments, the imaging system 106 may be configured to obtain one or more motion/measurement scans (“M-scans”) based on multiple A-scans. In particular, an OCT M-scan may be a one-dimensional profile of the optical scattering intensity of the target tissue 110 as a function of depth that may be constructed from multiple A-scans taken at a single point within the target tissue 110 over time. The result may be a two-dimensional graph that represents the reflectivity of different tissue layers along the depth axis over time. In some embodiments, an M-scan may be used to capture the deformation in the target tissue 110 due to applied forces. For example, an M-scan may be used to capture the deformation in the target tissue 110 over time such that OCE may be used to determine biomechanical properties. In the present disclosure, reference to an “M-scan” may refer to the process of performing multiple A-scans as well as the one-dimensional profile that may be constructed from the corresponding A-scans.

The patient interface 114 may include a mount that is configured to engage with the target tissue 110 to hold the target tissue 110 in position during performance of the ophthalmic procedure. In these and other embodiments, the patient interface 114 may be configured to allow the laser beam to pass therethrough to allow for performance of the procedure via the laser beam.

In some embodiments, the patient interface 114 may include a soft-fit insert (“insert”) that may be similar to a contact lens. In these and other embodiments, the insert may be installed on an optical surface (e.g., a lens) of the patient interface 114 such that the insert may be within an optical measurement path used to perform optical imaging of the target tissue 110. For example, while the imaging system 106 is illustrated adjacent to the patient interface 114, it will be appreciated that the imaging system 106 may sense or otherwise image the target tissue 110 through the patient interface 114. Additionally or alternatively, the insert may be moistened (e.g., with a saline solution) to help maintain flexibility of the insert, which may be such that a liquid layer may be disposed on at least one surface of the insert. In some instances, the liquid layer may accordingly be disposed in an optical measurement path of light that may be measured and used for optical imaging (“measurement light”).

In some embodiments, deformations, displacements, waves, and/or micro-motion may be induced in the target tissue 110 while the imaging system 106 captures OCT images of the target tissue 110 such that OCE may be performed on the captured OCT images. In some embodiments, the deformations, displacements, waves, and/or micro-motion in the target tissue 110 may be induced mechanically, acoustically, and/or optically. In some of these embodiments, the laser 102 may induce deformations, displacements, waves, and/or micro-motion in the target tissue 110 enabling OCE to be performed to determine one or more biomechanical properties of the target tissue 110. In some embodiments, deformations, displacements, waves, or micro-motion may be induced in the target tissue 110 via a probe, a compression plate, a vibrating source (e.g. an actuator or motor), a magnet, a microheater, and/or a transducer.

The tissue analysis module 112 may include any suitable system, apparatus, or device, configured to perform one or more tissue analysis operations with respect to the target tissue 110. For example, in some embodiments, the tissue analysis module 112 may include code and routines configured to allow a computing system to perform one or more tissue analysis operations. Additionally or alternatively, the tissue analysis module 112 may be implemented using hardware including one or more processors, CPUs graphics processing units (GPUs), data processing units (DPUs), parallel processing units (PPUs), microprocessors (e.g., to perform or control performance of one or more operations), field-programmable gate arrays (FPGA), application-specific integrated circuits (ASICs), accelerators (e.g., deep learning accelerators (DLAs)), one or more programmable vision accelerators (PVAs), which may include one or more vector processing units (VPUs), one or more direct memory access (DMA) systems, one or more pixel processing engines (PPEs), etc., and/or other processor types. In these and other embodiments, the tissue analysis module 112 may be implemented using a combination of hardware and software.

In some embodiments, the tissue analysis module 112 may include an artificial intelligence model such as a machine learning model, a supervised learning model (e.g., regression model, classification model, tree-based model), an unsupervised model (e.g., clustering model), and/or a deep learning model (e.g., convolutional neural networks, recurrent neural networks, transformer models), among others. In some embodiments, a separate computing system may include an artificial intelligence model and the tissue analysis module 112 may direct performance of operations by the artificial intelligence model on the separate computer system. In the present disclosure, operations described as being performed by the tissue analysis module 112 may include operations that the tissue analysis module 112 may direct a corresponding computing system to perform. In these or other embodiments, the tissue analysis module 112 may be implemented by one or more computing systems, such as that described in further detail with respect to FIG. 7 of the present disclosure.

In some embodiments, the tissue analysis module 112 may be configured to analyze the images obtained from the imaging system 106 to determine one or more biomechanical properties of the target tissue 110 and/or one or more geometric properties of the target tissue 110. For example, the tissue analysis module 112 may be configured to analyze A-scans, B-scans, C-scans, and/or M-scans obtained by the imaging system 106 to determine one or more geometric properties (e.g., dimensions, size, structure, and/or, shape) of a cataract. In some embodiments, the tissue analysis module 112 may perform functional extensions of OCT imaging such as OCE on the images obtained from the imaging system 106 to determine one or more biomechanical properties of the target tissue 110. For example, the tissue analysis module 112 may be configured to analyze A-scans, B-scans, C-scans, and/or M-scans obtained by the imaging system 106 to determine one or more biomechanical properties (e.g., elasticity, stiffness, and/or viscoelasticity) of a cataractous lens using OCE. In some embodiments, the tissue analysis module 112 may perform post-processing techniques such as speckle decorrelation and/or OCT phase decorrelation on the OCT scans. In some embodiments, the tissue analysis module 112 may perform light attenuation analysis based on OCT images obtained from the imaging system 106. In these and other embodiments, the tissue analysis module 112 may determine one or more biomechanical properties such as opacity from the light attenuation of the OCT signals in the OCT images. For example, the tissue analysis module 112 may perform light attenuation analysis on A-scans, B-scans, C-scans, and/or M-scans obtained by the imaging system 106 to determine opacity.

In these and other embodiments, the tissue analysis module 112 may be configured to determine one or more biomechanical properties of the target tissue 110 corresponding to an eye based on an optical imaging result obtained using an optical imaging system such as the imaging system 106. In some embodiments, the tissue analysis module 112 may be configured to perform OCE and/or light attenuation analysis based on OCT images obtained from the imaging system 106 to determine one or more biomechanical properties.

Additionally or alternatively, the tissue analysis module 112 may be configured to determine one or more geometric properties of the target tissue 110 based on an optical imaging result obtained using an optical imaging system such as the imaging system 106. For example, the tissue analysis module 112 may determine one or more geometric properties of the target tissue 110 based on OCT images of the target tissue 110 obtained utilizing the imaging system 106.

In some embodiments, the tissue analysis module 112 may be configured to generate a customized treatment plan for treatment of the target tissue 110 based on the one or more biomechanical properties and/or the one or more geometric properties of the target tissue 110. In some embodiments, the customized treatment plan may pertain to femtosecond laser-assisted cataract surgery. In these and other embodiments, the customized treatment plan may include a three-dimensional plan for removing at least a portion of the target tissue 110. For example, the customized treatment plan may include a three-dimensional plan for removing at least a portion of a cataractous lens of an eye. Reference to the removal of at least a portion of the target tissue 110 throughout this disclosure may include the formation of microcavitation bubbles in the target tissue 110. In these and other embodiments, the customized treatment plan may include one or more surgical parameters corresponding to at least a portion of the target tissue 110. In some of these embodiments, the surgical parameters may be determined based on the one or more biomechanical properties and/or the one or more geometric properties. In some embodiments, the customized treatment plan may include different laser parameters for the laser 102 for different portions of a cataractous lens depending on the biomechanical properties of the cataractous lens at those different portions. For example, the laser intensity, power, duration, and/or trajectory may vary depending on the stiffness and/or opacity of the cataractous lens. For example, the laser intensity, laser power, and laser duration of application may be higher in regions of higher stiffness and opacity than for regions of lower stiffness and opacity.

Thus, the laser 102 may be adjusted depending on whether the biomechanical properties indicate that the portion of the cataractous lens requires more energy to fragment (e.g., higher stiffness, higher opacity, higher light attenuation, higher light scattering intensity, lower transparency, higher grade, higher severity, lower strain, and/or lower Poisson's ratio) or less energy to fragment (e.g., lower stiffness, lower opacity, lower light attenuation, lower light scattering intensity, higher transparency, lower grade, lower severity, higher strain, and/or higher Poisson's ratio).

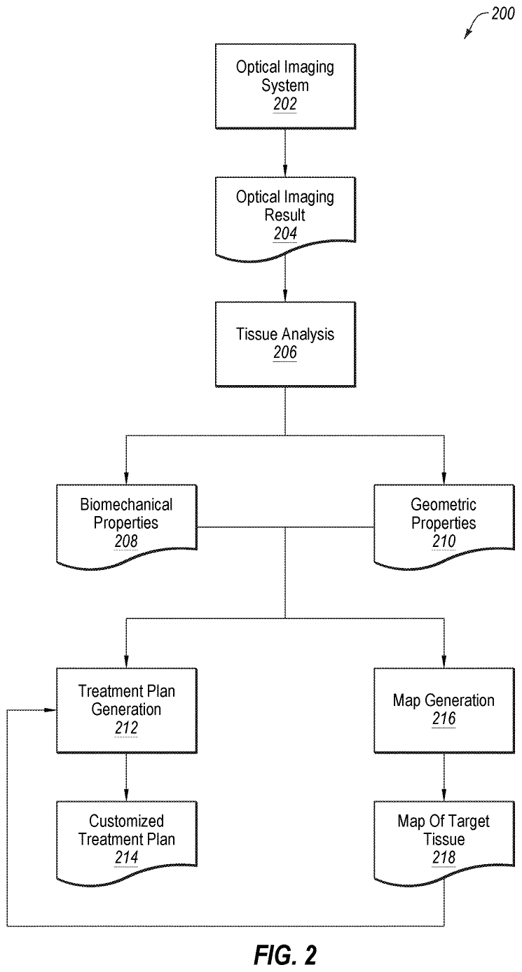

In these and other embodiments, the tissue analysis module 112 may generate a map of the target tissue 110 in three-dimensional space based on the one or more biomechanical properties and/or the one or more geometric properties. In some of these embodiments, the customized treatment plan may be generated based on the map of the target tissue 110. For example, the tissue analysis module 112 may be configured to map the tissue scattering of the target tissue 110 in three-dimensions to determine the customized treatment plan. In some embodiments, the map of the target tissue 110 may be generated in two-dimensional space based on the one or more biomechanical properties and/or the one or more geometric properties. In these and other embodiments, the map of the target tissue 110 may allow for the size, shape, and dimensions of the target tissue 110 such as a cataractous lens to be determined. In some embodiments, the biomechanical properties determined by the tissue analysis module 112 may be included in the map of the target tissue 110. For example, the map of the target tissue 110 may include the stiffness of a cataract. Further details of the map generated by the tissue analysis module 112 are described with respect to FIG. 2-FIG. 5.

In some embodiments, the tissue analysis module 112 may identify an altered portion of the target tissue 110 based on an optical imaging result obtained using an optical imaging system such as the imaging system 106 compared to a previous optical imaging result of the target tissue 110. For example, the tissue analysis module 112 may identify a portion of a cataractous lens that has been removed in substantially real-time as a surgery is performed based on an OCT image received during surgery from the imaging system 106.

In some embodiments, the tissue analysis module 112 may update the map of the target tissue 110 in substantially real-time to reflect the altered portion of the target tissue 110. For example, the tissue analysis module 112 may identify a cataractous lens that has been modified compared to a previous optical imaging result (e.g., a portion of the cataractous lens has been removed during surgery), and the tissue analysis module 112 may generate an updated map of the target tissue 110 to reflect that a portion of the cataractous lens has been removed. As another example, the updated map may reflect that the cataractous lens has been broken apart into multiple pieces, softened (e.g., the density broken up), or otherwise processed. Because the tissue analysis module 112 may receive the optical imaging results from the imaging system 106, there may be a small lag between when the optical imaging result is received and when the map of the target tissue 110 is generated, however, the lag may be due to processing by the tissue analysis module 112 and delays corresponding to the lag may still be considered as being part of “real-time.”

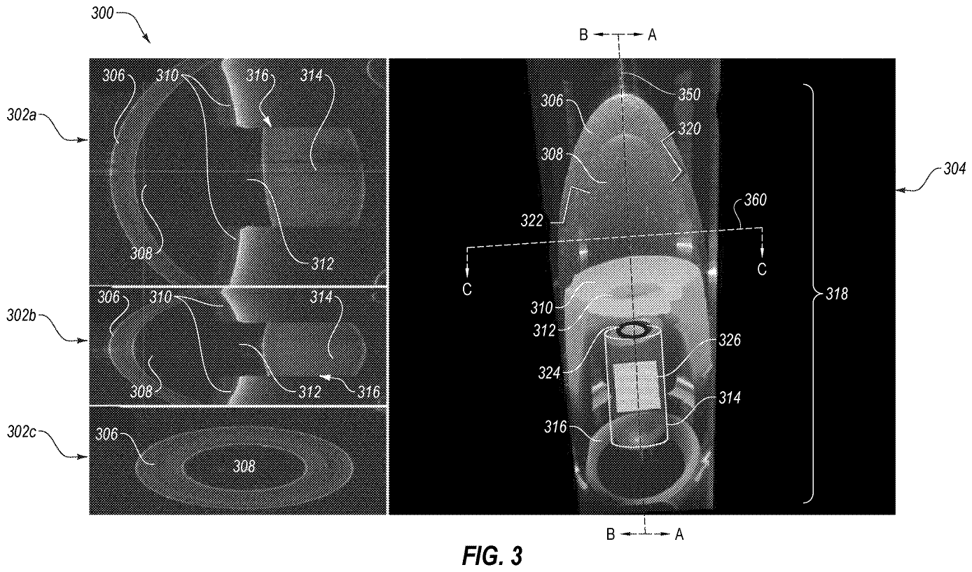

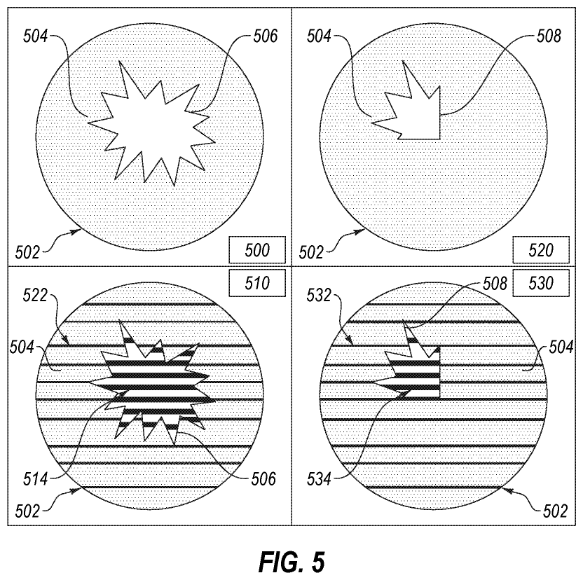

In these and other embodiments, the tissue analysis module 112 may update the customized treatment plan based on the updated map of the target tissue 110. For example, the tissue analysis module 112 may adjust the recommended laser parameters of the laser 102 in the customized treatment plan based on the updated map. For example, the laser power, intensity, and/or duration may be adjusted based on an updated map reflecting the tissue cuttings during fragmentation of the cataract. Furthermore, the biomechanical properties (e.g., stiffness and opacity) may be redetermined during fragmentation of the cataractous lens, and the map and treatment plan may be updated based on the updated biomechanical properties. Thus, the tissue analysis module 112 may update the customized treatment plan dynamically in real-time to adjust various components of the treatment plan such that the surgery may be performed more efficiently, effectively, and safely. Additional details of updating the map are provided in the description with respect to FIG. 2, FIG. 3, and FIG. 5.

In some embodiments, the tissue analysis module 112 may be configured to cause an artificial intelligence model (not illustrated) to determine the one or more biomechanical properties, determine the one or more geometric properties, generate a map of the target tissue 110 in three-dimensional space based on the one or more biomechanical properties and the one or more geometric properties, and/or generate the customized treatment plan. In these and other embodiments, the tissue analysis module 112 may cause the artificial intelligence model to identify the altered portion of the target tissue 110 or update the customized treatment plan.

In some embodiments, the tissue analysis module 112 may access an image database. In some of these embodiments, the image database may include multiple images obtained from the imaging system 106. In these and other embodiments, the image database may include a repository of OCT images that may be utilized to train an artificial intelligence model included in the tissue analysis module 112 or separate from the tissue analysis module 112. In some embodiments, the image database may also include biomechanical property data and/or geometric property data of the target tissues corresponding to each of the images. In some embodiments, the image database may be included on a separate device.

The system control module 108 may include any suitable system, apparatus, or device, configured to perform one or more control operations with respect to the system 100. For example, in some embodiments, the system control module 108 may include code and routines configured to allow a computing system to perform one or more operations. Additionally or alternatively, the system control module 108 may be implemented using hardware including one or more processors, CPUs graphics processing units (GPUs), data processing units (DPUs), parallel processing units (PPUs), microprocessors (e.g., to perform or control performance of one or more operations), field-programmable gate arrays (FPGA), application-specific integrated circuits (ASICs), accelerators (e.g., deep learning accelerators (DLAs)), one or more programmable vision accelerators (PVAs), which may include one or more vector processing units (VPUs), one or more direct memory access (DMA) systems, one or more pixel processing engines (PPEs), etc., and/or other processor types. In these and other embodiments, the system control module 108 may be implemented using a combination of hardware and software.

In some embodiments, the system control module 108 may include an artificial intelligence model such as a machine learning model, a supervised learning model (e.g., regression model, classification model, tree-based model), an unsupervised model (e.g., clustering model), and/or a deep learning model (e.g., convolutional neural networks, recurrent neural networks, transformer models), among others. In some embodiments, a separate computing system may include an artificial intelligence model and the system control module 108 may direct performance of operations by the artificial intelligence model on the separate computer system. In the present disclosure, operations described as being performed by the system control module 108 may include operations that the system control module 108 may direct a corresponding computing system to perform. In these or other embodiments, the system control module 108 may be implemented by one or more computing systems, such as that described in further detail with respect to FIG. 7 of the present disclosure.

In some embodiments, the system control module 108 may be configured to control the laser 102, the optics module 104, and/or the tissue analysis module 112. Additionally or alternatively, the system control module 108 may be configured to control any number of other components of the system 100 not expressly illustrated.

In some embodiments, the system control module 108 may be configured to determine the placement of the laser beam and corresponding laser pulses with respect to the target tissue 110. In these and other embodiments, the system control module 108 may be configured to determine the placement of the laser beam and the corresponding laser pulses based on one or more optical images.

In instances where the customized treatment plan is generated by the tissue analysis module 112, the system control module 108 may be configured to adjust the laser 102 according to the customized treatment plan. For example, the customized treatment plan generated by the tissue analysis module 112 may include one or more surgical parameters corresponding to a cataractous lens and the laser 102 may be adjusted by the system control module 108 depending on the laser parameters of the customized treatment plan. Additionally or alternatively, in instances where the customized treatment plan is updated by the tissue analysis module 112, the system control module 108 may be configured to adjust the laser 102 according to the updated customized treatment plan.

In instances where the customized treatment plan includes one or more surgical parameters, the system control module 108 may adjust components of the system 100 based on the surgical parameters. For example, the customized treatment plan may include surgical parameters such as a cutting location, a cutting trajectory, and/or a cutting depth and the system control module 108 may adjust the laser 102 to perform cuts corresponding to the cutting location, the cutting trajectory, and/or the cutting depth in the customized treatment plan. In another example, the customized treatment plan may include a laser parameter such as laser intensity, laser power, laser duration, and/or laser trajectory and the system control module 108 may adjust the laser 102 such that the laser intensity, laser power, laser duration, and/or laser trajectory are implemented according to the customized treatment plan.

In some embodiments, the system control module 108 may control one or more actuators of the optics module 104 to adjust a location of the laser beam and corresponding laser pulses. Additionally or alternatively, the system control module 108 may adjust a pattern of the laser beam through adjustment of the laser 102 and/or the optics module 104. In these and other embodiments, the system control module 108 may adjust one or more other actuators that may move the entire system 100 or the laser 102 itself such that the laser orientation may be moved with respect to the target tissue 110. Additionally or alternatively, the system control module 108 may cause the adjustment of a support platform (e.g., bed) that the patient is lying on to adjust the orientation of the laser 102 with respect to the target tissue 110 (e.g., by controlling one or more actuators corresponding to the support platform).

Modifications, additions, or omissions may be made to FIG. 1 without departing from the scope of the present disclosure. For example, the system 100 may include more or fewer elements depending on the implementation. Further, the system 100 may be configured to perform any number of operations as compared to those explicitly described. In addition, the principles described may be applied to any applicable optical imaging result and are not limited to OCT imaging, OCE, or light attenuation analysis.

FIG. 2 illustrates an example process 200 that may be performed to generate a customized treatment plan 214, according to one or more embodiments of the present disclosure. Each operation or block of the process 200 described herein, may comprise a computing process that may be performed using any combination of hardware, firmware, and/or software. For instance, various functions may be carried out by a processor executing instructions stored in memory. The process 200 may also be embodied as computer-usable instructions stored on computer storage media. The process 200 may be provided by a standalone application, a service or hosted service (standalone or in combination with another hosted service), as a microservice via an application programming interface (API) or a plug-in to another product, to name a few. In addition, the process 200 is described, by way of example, with respect to the system of FIG. 1. However, the process 200 may additionally or alternatively be executed by any one system, or any combination of systems, including, but not limited to, those described herein. Further, to ease explanation, the description of the process 200 is given with respect to generating a customized treatment plan corresponding to treatment of a cataractous lens of an eye, however such a process may be used for the generation of a customized treatment plan for any number of other target tissues.

The process 200 may include obtaining an optical imaging result 204 from an optical imaging system 202. In some embodiments, the optical imaging system 202 may be the same or similar to the imaging system 106 described with reference to FIG. 1. In some embodiments, the optical imaging system 202 may include any suitable system, apparatus, or device, that may be configured to obtain one or more optical imaging results 204 of the eye corresponding to a target tissue (such as the target tissue 110 of FIG. 1). For example, in some embodiments, the optical imaging system 202 may collect reflected or scattered light or sound from the target tissue to capture image data corresponding to the target tissue.

The optical imaging system 202 may include one or more different types of devices and/or systems configured to capture various different types of images of the eye as corresponding to the target tissue. For example, in some embodiments, the optical imaging system 202 may include a camera and the optical imaging result 204 may be a captured camera image of the eye. In these and other embodiments, the optical imaging system 202 may include an ultrasound imaging device and the optical imaging result 204 may be a captured ultrasound image of the eye. Additionally or alternatively, the optical imaging system 202 may include an OCT device.

In these and other embodiments, the optical imaging result 204 may be an OCT scan such as an A-scan, a B-scan, a C-scan, or an M-scan. In these and other embodiments, the optical imaging result 204 may include OCE results and/or light attenuation analysis results. For example, the optical imaging result 204 may include deformation, wave, and/or micro-motion data and/or light attenuation data. In these and other embodiments, the optical imaging result 204 may be an image of an eye, a cataractous lens of an eye, and/or a cataract of an eye. In some embodiments, the optical imaging result 204 may be a two-dimensional image or a three-dimensional image.

In some embodiments, the process 200 may include storing the optical imaging result 204 obtained from the optical imaging system 202 in an image database. In some of these embodiments, the image database may include multiple optical imaging results 204 obtained from the optical imaging system 202. In these and other embodiments, the image database may include a repository of OCT images that may be utilized to train an artificial intelligence model. In these and other embodiments, the image database may also include biomechanical property data and/or geometric property data of the target tissues corresponding to each of the optical imaging results 204.

As illustrated in FIG. 2, in some embodiments, the process 200 may include a tissue analysis operation 206 (“tissue analysis 206”) configured to determine the biomechanical properties 208 and/or the geometric properties 210 of the target tissue based on the optical imaging result 204. For example, the biomechanical properties 208 and/or the geometric properties 210 of a cataractous lens may be determined from an OCT imaging result. In some embodiments, the biomechanical properties 208 may include opacity, transparency, elasticity, viscoelasticity, stiffness, strain, Poisson's Ratio, density, light scattering intensity, light attenuation, shear modulus, grading of the target tissue, nuclear severity, cortical severity, posterior subcapsular severity, and type of target tissue. In some embodiments, the geometric properties 210 may include the size, shape, structure, and/or dimensions of the target tissue.

In some embodiments, the biomechanical properties 208 may be determined from OCT images. For example, opacity of a cataractous lens may be determined from the light scattering intensity in an OCT image of an eye. In some embodiments, the biomechanical properties 208 may be determined from functional extensions of OCT such as OCE and/or light attenuation analysis. For example, elasticity, stiffness, strain, Poisson's ratio, shear modulus, and/or viscoelasticity of a cataractous lens may be determined from OCE analysis based on an OCT image of an eye. In another example, opacity, stiffness, transparency, and/or density of a cataractous lens may be determined from light attenuation analysis based on an OCT image of an eye.

In some embodiments, the tissue analysis 206 may include determining one or more biomechanical properties 208 from other determined biomechanical properties 208. For example, grade of the target tissue, nuclear severity, cortical severity, and/or posterior subcapsular severity may be determined based on density and/or opacity. For instance, the nuclear severity of a cataract may be determined based on the density of the cataract in that the higher the density (or light scattering) the greater the determined severity and vice versa. In these and other embodiments, the tissue analysis 206 may determine the grading of the cataract from the nuclear severity, the cortical severity, and/or the posterior subscapular severity.

In some embodiments, the tissue analysis 206 may include measuring backscattered light from the optical imaging result 204. In some of these embodiments, the optical imaging result 204 may be an OCT scan of an eye and the tissue analysis 206 may include reconstructing the profile of the eye at different depths based on the backscattered light. In these and other embodiments, the tissue analysis 206 may include determining one or more geometric properties 210 of the target tissue based on the reconstructed profile of the eye such as the dimensions, size, structure, and/or shape of the target tissue. For example, the dimensions, size, structure, and/or shape of the cataractous lens may be determined by reconstructing the profile of the eye (including the cataractous lens) at different depths based on the backscattered light.

In some embodiments, the tissue analysis 206 may include segmenting the optical imaging result 204 to analyze the target tissue. For example, the optical imaging result 204 may be an image of an eye with a cataractous lens and the tissue analysis 206 may segment the optical imaging result 204 in order to analyze the cataractous lens of the eye. In some embodiments, the tissue analysis 206 may further segment the optical imaging result 204 to analyze specific portions of the target tissue. For example, the optical imaging result 204 may be further segmented to analyze the cataract and/or further segmented to analyze specific portions of the cataract in order to determine biomechanical properties 208 and/or geometric properties 210 in the specific portions.

In some embodiments, the tissue analysis 206 may include performing OCE on the optical imaging result 204 to determine one or more biomechanical properties 208. In these embodiments, OCE may be performed on OCT scans based on local micro-motion, deformation, and/or waves induced in the target tissue and the associated OCT signal variation as a result of the micro-motion, deformation, and or waves induced in the target tissue. In some embodiments, the tissue analysis 206 may include post-processing techniques such as speckle decorrelation or OCT phase decorrelation. In some embodiments, the biomechanical properties 208 determined from OCE in the tissue analysis 206 may include determining elasticity of the target tissue, viscoelasticity of the target tissue, strain of the target tissue, stiffness of the target tissue, Poisson's ratio of the target tissue, shear modulus, and/or density of the target tissue either directly or indirectly from the displacements, deformations, micro-motion, and/or waves induced in the target tissue.

In some embodiments, the tissue analysis 206 may include performing light attenuation analysis on the optical imaging result 204 to determine one or more biomechanical properties 208. In at least some of these embodiments, light attenuation may be determined from an OCT signals in an OCT scan. For example, light attenuation may be determined from multiple A-scans. In some embodiments, the biomechanical properties 208 determined from light attenuation analysis may include light attenuation of the target tissue, opacity of the target tissue, stiffness (or hardness) of the target tissue, transparency of the target tissue, and/or density of the target tissue.

In some embodiments, the light attenuation may be calculated as a relative ratio of attenuation along a light path. For example, a light attenuation coefficient may be determined based on the following equation where I is OCT signal intensity in linear space, i is pixel index in each A-scan, and A is the pixel size. In some embodiments, the light attenuation coefficient may be used as a metric or a proxy for opacity, density, stiffness, strain, and/or elasticity. In these and other embodiments, the inverse of the light attenuation coefficient may be used as a metric or a proxy for transparency. The equation represented below demonstrates one technique to determine the light attenuation coefficient, but other techniques may be sufficient.

$$Light Attenuation [ i ] = \frac{I [ i ]}{2 Δ {\sum }_{( i + 1 )}^{∞} I [ i ]}$$

In some embodiments, the process 200 may further include a map generation operation 216 (“map generation 216”) configured to generate a map of the target tissue 218. In some embodiments, the map of the target tissue 218 may be a 3D map based on the biomechanical properties 208 and/or the geometric properties 210 determined by the tissue analysis 206. For example, the map generation 216 may generate a map of a cataractous lens that may provide 3D views of the cataractous lens and the biomechanical properties 208 of the cataractous lens to determine removal strategies. In some embodiments, the map generation 216 may generate 2D maps of the target tissue and/or 3D maps of the target tissue to aid in visualization of the target tissue. Further example details with respect to the map of the target tissue 218 are discussed in more detail with respect to the 2D maps 302 and the 3D map 304 described with respect to FIG. 3

In some embodiments, the map generation 216 may generate the map of the target tissue 218 from the optical imaging result 204. In these and other embodiments, the map generation 216 may utilize an OCT image to map the tissue scattering of the target tissue in 3D. In these and other embodiments, the map generation 216 may utilize various 2D images of the target tissue to generate the map of the target tissue 218 in 3D.

In some embodiments, the process 200 may further include a treatment plan generation operation 212 (“treatment plan generation”) configured to generate a customized treatment plan 214. In some embodiments, the treatment plan generation 212 may be performed by any suitable system, apparatus, or device, that may be configured to generate the customized treatment plan 214 based on the biomechanical properties 208 and/or geometric properties 210 of an eye corresponding to a target tissue (such as the target tissue 110 of FIG. 1). For example, the customized treatment plan 214 may be generated by the tissue analysis module 112 of FIG. 1.

In some embodiments, the treatment plan generation 212 may include determining one or more surgical parameters based on the biomechanical properties 208 and/or the geometric properties 210 for inclusion in the customized treatment plan 214. In these embodiments, the surgical parameters may include a laser parameter, a cutting location, a cutting trajectory, a cutting depth, ultrasound energy, or surgical tool selection. In instances where laser parameters are included, the laser parameters may include laser intensity, laser power, laser duration, and/or laser trajectory. In some embodiments, the surgical parameters may pertain to FLACS. In some embodiments, the laser parameters may pertain to a femtosecond laser.

In some embodiments, the surgical parameters may correspond to a portion of the target tissue and may vary according to the biomechanical properties 208 corresponding to the portion of the target tissue. For example, the laser intensity, power, and duration determined by the treatment plan generation 212 may vary according to the stiffness of the cataractous lens in that the laser intensity, power, and duration may be higher for stiffer regions of a cataractous lens than for regions of the cataractous lens that are not as stiff In another example, the cutting locations, depth, and trajectories may vary depending on the grade or severity of the cataractous lens (e.g., nuclear severity, cortical severity, posterior subcapsular severity) in that different fragmentation strategies of the cataractous lens may be implemented depending on these determined biomechanical properties 208. For example, different cutting locations, cutting depths, and cutting trajectories may be utilized for different cataractous lenses based on the biomechanical properties 208 of the particular cataractous lens. The determination of the surgical parameters based on the biomechanical properties 208 and/or the geometric properties 210 is discussed in further detail with respect to FIG. 3-FIG. 5.

In some embodiments, the treatment plan generation 212 may place different weights on different biomechanical properties 208 and/or geometric properties 210 to determine various surgical parameters for inclusion in the customized treatment plan 214. For example, the stiffness of a cataractous lens may be given a higher weight than opacity of a cataractous lens. Thus, the laser intensity included in the customized treatment plan 214, for example, may be increased more for a stiffer region of a cataractous lens than the laser intensity would be for a more opaque region of the cataractous lens.

In some embodiments, the treatment plan generation 212 may include generation of a 3D plan for inclusion in the customized treatment plan 214. In some embodiments, the 3D plan may be a plan for removing at least a portion of the target tissue. For example, the treatment plan generation 212 may generate a 3D plan for removing a cataractous lens in an eye (which, as described previously, may include the formation of microcavitation bubbles in the target tissue). In some embodiments, the 3D plan may be generated based on the geometric properties 210 and/or the biomechanical properties 208 of the target tissue. In some embodiments, the treatment plan generation 212 may utilize the map of the target tissue 218 to generate the customized treatment plan 214. For example, the treatment plan generation 212 may utilize a map of a cataractous lens to generate a 2D and/or 3D plan for removing the cataractous lens. In these and other embodiments, OCT may be utilized to map the tissue scattering in 3D and the surgical parameters may be mapped to the 3D map of the tissue scattering to illustrate cutting locations, cutting trajectories, cutting depths, surgical tool selection, ultrasound energy, and/or laser parameters corresponding to different regions of the target tissue. For instance, a three-dimensional plan for FLACS may be generated by the treatment plan generation 212 demonstrating the incisions on the cornea, the capsulotomy incision, and the fragmentation strategy for the cataractous lens in three-dimensional space. Additional details with respect to the 3D plan that may be included in the customized treatment plan 214 are described with respect to FIG. 3.

In these and other embodiments, the treatment plan generation 212 may include generation of 2D plans for removing at least a portion of the target tissue for inclusion in the customized treatment plan 214. In some embodiments, the 2D plans may correspond to horizontal slices of the 3D plan generated by the treatment plan generation 212. For example, the treatment plan generation 212 may generate a 3D plan for removing a cataractous lens in an eye and the treatment plan generation 212 may also generate 2D slices corresponding to different layers of the cataractous lens or different layers of the eye. In these embodiments, the surgical parameters may vary depending on the 2D plan according to the biomechanical properties 208 and/or geometric properties 210. In some embodiments, the 2D plan generated by the treatment plan generation 212 may be a 2D plan for FLACS. In these and other embodiments, the 2D plan may pertain to fragmentation of a cataractous lens. Additional details with respect to the 2D plan that may be included in the customized treatment plan 214 are described with respect to FIG. 3-FIG. 5.

As a result of the treatment plan generation 212, the removal of the cataractous lens may be improved in that the cataractous lens may be more efficiently fragmented and removed by utilizing surgical parameters based on the biomechanical properties 208 and/or geometric properties 210 of the cataractous lens rather than according to generic treatment plans. Furthermore, the treatment plan generation 212 may allow for safer surgery with less risk to the patient in that surgery time may be reduced, and/or laser dosage and/or duration may be improved according to the biomechanical properties 208 and/or geometric properties 210 of the target tissue.

In some embodiments, the process 200 may be performed sequentially with additional optical imaging results of the target tissue to update the customized treatment plan 214, update the map of the target tissue 218, generate a new customized treatment plan, and/or generate a new map of the target tissue. For example, in some embodiments, one or more additional optical imaging results of a cataractous lens may be obtained from the optical imaging system 202 during surgery. Tissue analysis 206 may be performed while surgery is ongoing to determine biomechanical properties 208 and/or geometric properties 210 of the cataractous lens. In some embodiments, the tissue analysis 206 may identify that part of the target tissue has been changed, modified, or otherwise altered. For example, the tissue analysis 206 may determine that a portion of the cataractous lens has been removed (e.g., part of a cataractous lens has been removed during FLACS, e.g., due to fragmentation or other treatment of the cataractous lens). In another example, the tissue analysis 206 may determine that the biomechanical properties 208 and/or geometric properties 210 are different than those determined based on the optical imaging result 204. For example, the tissue analysis 206 may determine that the stiffness in a portion of a cataractous lens is different in the optical imaging result received during surgery than the stiffness in an optical imaging result received before surgery.

In some embodiments, the map generation 216 may update the map of the target tissue 218 to reflect the current state of the target tissue based on the most recently received optical imaging result. For example, the map generation 216 may update the map of the target tissue 218 to reflect that a portion of the cataractous lens has been removed. In some of these embodiments, the updating of the map of the target tissue 218 may be performed in substantially real-time such that the map of the target tissue 218 may be updated during surgical operations.

In some of these embodiments, the treatment plan generation 212 may update the customized treatment plan 214 based on the updated map of the target tissue 218. For example, the map of the target tissue 218 may be updated based on the tissue analysis 206 determining a different stiffness at a specific region of a cataractous lens, and the customized treatment plan 214 may adjust the laser intensity and/or laser power according to the updated stiffness. Additionally or alternatively, a 2D or 3D plan for removing the cataractous lens may be updated based on the updated map of the target tissue. This may, for example, allow for surgical parameters included in the customized treatment plan 214 to be adjusted during surgery based on optical imaging results received during surgery.

While described as being performed during surgery, it will be appreciated that the updating of the customized treatment plan 214 and/or the updating of the map of the target tissue 218 may be performed pre-surgery, in some embodiments. Further details regarding the updating of the map of the target tissue 218 and the updating of the customized treatment plan 214 based on multiple optical imaging results are described with reference to FIG. 5.

Modifications, additions, or omissions may be made to FIG. 2 without departing from the scope of the present disclosure. For example, the process 200 may include more or fewer operations depending on the implementation. In addition, the principles described may be applied to any applicable optical imaging result and are not limited to OCT imaging, OCE, or light attenuation analysis.

In addition, one or more of the operations described with respect to FIG. 2 may be performed by an artificial intelligence (AI) model. For example, in some embodiments, the AI model may perform the tissue analysis 206 and any operations associated therewith. In some embodiments, the AI model may be used for determining the one or more biomechanical properties 208 based on the optical imaging result 204. In some embodiments, the AI model may be used for determining the geometric properties 210 based on the optical imaging result 204. In some embodiments, the AI model may perform the treatment plan generation 212 and any operations associated therewith. For example, the AI model may generate the customized treatment plan 214.

In some embodiments, one or more optical imaging results 204 may be provided to the AI model as training data. In some of these embodiments, the optical imaging results 204 may be images of cataractous lenses, which have been segmented from OCT images of an eye. In these and other embodiments, the optical imaging results 204 may include associated biomechanical properties 208 such as cataract grade, type, and density. In some embodiments, the AI model may be trained using the optical imaging results 204 such that, when another optical imaging result is provided to the AI model, the AI model may evaluate the optical imaging result to determine the biomechanical properties 208 of a cataractous lens depicted in the optical imaging result such as the cataract grade, type, severity, and density, for example. In these embodiments, the AI model may also determine surgical parameters such as cutting locations, cutting depth, cutting trajectory, laser parameters, ultrasound energy, and/or surgical tool selection based on the optical imaging result.

In some embodiments, the AI model may identify an altered portion of the target tissue based on another optical imaging result. In some embodiments, the AI model may generate or update the customized treatment plan 214 based on the map of the target tissue 218, which may be based on the optical imaging result.

In addition, the use cases for generating the customized treatment plan 214 may not be limited to only optical treatment plans. For example, the generation of a customized treatment plan based on one or more biomechanical properties and/or geometric properties of the target tissue 110 may be applicable to imaging of any suitable target tissue where these properties may improve the treatment plan.

FIG. 3 illustrates an example of a customized treatment plan 300, according to one or more embodiments of the present disclosure. In some embodiments, and as illustrated in FIG. 3, the example customized treatment plan 300 may include one or more 2D maps 302a-302c (collectively 2D maps 302) of an eye and one or more 3D maps 304 of the eye. As illustrated in FIG. 3, the 2D maps 302 and the 3D map 304 may illustrate a cornea 306, an anterior chamber 308, an iris 310, a pupil 312, a cataractous lens 314, and a capsular bag 316.

In some embodiments, the 2D maps 302 and the 3D map 304 may be generated from one or more optical imaging results (e.g., the optical imaging result 204 described with reference to FIG. 2). In some embodiments and as illustrated in FIG. 3, the 2D maps 302 and the 3D map 304 may map tissue scattering from OCT images. The 2D and 3D visualization of the eye provided by the 2D maps 302 and the 3D maps 304 may provide a clearer understanding of the geometric properties of the eye. For example, the 3D map 304 may provide a clearer understanding of the size, shape, structure, and dimensions of the cataractous lens 314, thereby allowing for efficient, effective, and safer removal of the cataractous lens 314 based on the customized treatment plan 300.

In some embodiments and as illustrated in FIG. 3, the 2D maps 302 may correspond to a given horizontal slice of the 3D map 304. For example, the 2D map 302a illustrates a side, cross-sectional perspective of the eye along an A-side of a first cutting plane 350 illustrated on the 3D map 304, the 2D map 302b illustrates another side, cross-sectional perspective of the eye along a B-side of the first cutting plane 350 illustrated on the 3D map 304 (although skewed), and the 2D map 302c illustrates a top, cross-sectional perspective of the eye along a C-side of a second cutting plane 360 illustrated on the 3D map 304. The variety of perspectives included in the customized treatment plan 300 may allow a more effective removal plan of the cataractous lens 314 to be determined and implemented. The 2D maps 302 and the 3D map 304 are merely examples of 2D maps 302 and 3D maps 304 that may be generated from the one or more optical imaging results and are not necessarily anatomically correct as shown in FIG. 3 nor are the 2D maps 302 and 3D maps 304 necessarily to scale as shown in FIG. 3. For example, the 2D map 302b has been slightly compressed compared to the 2D map 302a. Additionally, the 2D map 302c has been slightly compressed such that the visualization of the eye is ovular rather than circular.

In some embodiments, the customized treatment plan 300 may include a 3D plan 318 for removal of the target tissue. For example and as illustrated in FIG. 3, the customized treatment plan 300 may include a 3D plan 318 for removal of the cataractous lens 314. In some embodiments, the customized treatment plan 300 may include one or more surgical parameters. In some embodiments, the surgical parameters may be included in the 3D plan 318. For example, cutting locations such as the primary incision 320 of the cornea 306, the secondary incision 322 of the cornea 306, the capsulotomy 324 of the capsular bag 316, and the fragmentation 326 of the cataractous lens may be included in the 3D plan 318.

As illustrated in FIG. 3, the 3D plan 318 may represent a cataract removal plan. In these embodiments, the primary incision 320 of the cornea 306 may be included in the 3D plan 318 and illustrated on the 3D map 304. The primary incision 320 of the cornea 306 may provide the main entry point for surgical instruments, a phacoemulsification probe, and for the replacement lens. The secondary incision 322 of the cornea 306 may also be included in the 3D plan 318 and illustrated on the 3D map 304. The secondary incision 322 of the cornea 306 may allow for secondary access to the eye to allow for irrigation, aspiration, and/or additional instruments. The capsulotomy 324 (a cut at an anterior portion of the capsular bag 316) may also be included in the 3D plan 318 and illustrated on the 3D map. The capsulotomy 324 may provide access into the capsular bag 316 such that the cataractous lens 314 may be removed while keeping a posterior portion of the capsular bag 316 intact so a replacement lens may be inserted into the capsular bag 316. The fragmentation 326 of the cataractous lens 314 may also be included in the 3D plan 318 and may be illustrated on the 3D map 304. The fragmentation 326 of the cataractous lens 314 may also require multiple cuts to segment the cataractous lens 314 into multiple pieces before the pieces may be emulsified using ultrasound energy (e.g., phacoemulsification). The fragmentation 326 strategy may be determined based on the biomechanical properties and/or geometric properties of the cataractous lens 314.

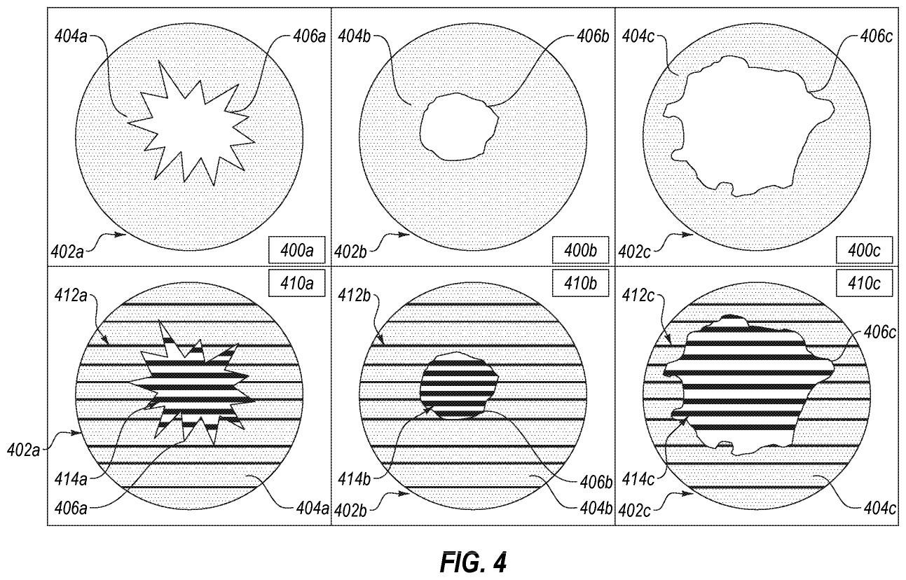

In some embodiments, the surgical parameters may vary depending on the position of the cutting locations on the 3D map 304. For example, the primary incision 320 may have a different cutting trajectory and cutting depth than the secondary incision 322. In some embodiments, different laser parameters, cutting trajectories, surgical tools, cutting depths, and/or ultrasound energy may be utilized at different cutting locations. In some embodiments, the surgical parameters may be based on the biomechanical properties and/or geometric properties of the target tissue (e.g., the biomechanical properties 208 and/or the geometric properties 210 of FIG. 2). For example, the fragmentation 326 of the cataractous lens 314 may also include multiple cutting locations and varying laser parameters, cutting depths, cutting trajectories, and/or ultrasound energies depending on the biomechanical properties and/or geometric properties of the cataractous lens 314. This is explained further with reference to FIGS. 4 and 5.

Modifications, additions, or omissions may be made to FIG. 3 without departing from the scope of the present disclosure. For example, the customized treatment plan 300 may omit one or more of the 2D maps 302 and/or the 3D map 304. In some embodiments, the 3D plan 318 may be omitted from the customized treatment plan 300. In some embodiments, the 3D plan 318 may include more or fewer cutting locations depending on the implementation. In addition, the principles described may be applied to any applicable optical imaging result and are not limited to OCT imaging, OCE, or light attenuation analysis. Furthermore, FIG. 3 describes an example customized treatment plan 300 pertaining to removal of the cataractous lens 314. In some embodiments, the customized treatment plan 300 may pertain to other ophthalmologic operations and/or non-ophthalmologic operations.

In addition, in some embodiments, the customized treatment plan 300 may be generated based on an optical imaging result (e.g., an OCT image of an eye) utilizing an AI model. For example, the 3D plan 318 may be generated based on an optical imaging result utilizing an AI model.

FIG. 4 illustrates example customized treatment plans 410a-410c based on optical imaging results 400a-400c of target tissues 402a-402c corresponding to cataractous lenses of various eyes. The customized treatment plan 410a may be generated based on the biomechanical properties and/or the geometric properties determined from the optical imaging result 400a of the target tissue 402a. The customized treatment plan 410b may be generated based on the biomechanical properties and/or the geometric properties determined from optical imaging result 400b of the target tissue 402b. The customized treatment plan 410c may be generated based on the biomechanical properties and/or the geometric properties determined from the optical imaging result 400c of the target tissue 402c. For example, the customized treatment plans 410a-410c may be generated according to the treatment plan generation operation 212 of process 200 described in FIG. 2. As illustrated in FIG. 4, each of the target tissues 402 may include an unclouded lens portion 404 (respectively unclouded lens portions 404a-404c) and various clouded lens portion 406 (respectively clouded lens portions 406a-406c). In some embodiments, the clouded lens portions 406 may be cataracts and the target tissues 402 may be cataractous lenses and may also be referred to as “clouded portions.”

As illustrated in FIG. 4, each of the customized treatment plans 410a-410c represents the fragmentation of the target tissues 402a-402c (e.g., the fragmentation 326 of the cataractous lens 314 described with reference to FIG. 3) mapped onto the optical imaging results 400a-400c. Each of the customized treatment plans 410a-410c may include a first set of surgical parameters 412a-412c respectively corresponding to the unclouded lens portions 404a-404c and a second set of surgical parameters 414a-414c respectively corresponding to the clouded lens portions 406a-406c. In some embodiments, the surgical parameters may include laser parameters (e.g., laser intensity, laser power, laser trajectory, laser duration), cutting depth, cutting location, cutting trajectory, ultrasound energy, and/or surgical tool selection. In some embodiments, the surgical parameters may pertain to FLACS. As illustrated in FIG. 4, the first sets of surgical parameters 412a-412c and the second sets of surgical parameters 414a-414c may include a cutting location (represented by the individual lines) and a laser intensity (represented by the thickness of the lines).

In operation, one or more biomechanical properties (e.g., the biomechanical properties 208 described with reference to FIG. 2) and/or geometric properties (e.g., the geometric properties 210 described with reference to FIG. 2) may be determined from the optical imaging results 400a-400c. Each of the target tissues 402a-402c may have different geometric properties (e.g., different sizes, shapes, and/or dimensions) and/or different biomechanical properties (e.g., elasticity, stiffness, viscoelasticity, opacity). For example, the clouded portion 406a has a star-like shape, the clouded portion 406b has a small rounded shape, and the clouded portion 406c has a large rounded shape. In another example, the target tissues 402a-402c may have different stiffnesses and/or opacities within the unclouded lens portions 404a-406c and different stiffnesses and/or opacities within the clouded lens portions 406a-406c. Based on, for example, the different stiffnesses, opacities, and/or shapes, the customized treatment plans 410a-410c may include different surgical parameters.

For example, the second set of surgical parameters 414a may have shorter cuts in the points of the star-like shape of the clouded lens portion 406a than the second set of surgical parameters 414a does in the body of the star-like shape of the clouded portion 406a. Furthermore, the second set of surgical parameters 414a may have a higher laser intensity (as illustrated by the thicker line) than the first set of surgical parameters 412a based on the determination that the clouded lens portion 406a is stiffer than the unclouded lens portion 404a.

In another example, the second sets of surgical parameters 414b and 414c may be different than the second set of surgical parameters 414a based on the different shapes and sizes of the clouded lens portions 406b and 406c, respectively. For example, the clouded lens portion 406c may have longer cuts than the clouded lens portions 406b and 406a due to the wider shape of the clouded lens portion 406c. Furthermore, the second sets of surgical parameters 414b and 414c may have different laser intensities than the second set of surgical parameters 414a because of the different biomechanical properties of the clouded lens portions 406b and 406c compared to the biomechanical properties of the clouded lens portion 406a. For example, the clouded lens portion 406b may be stiffer than the clouded lens portions 406a and 406c and, as a result, the customized treatment plan 410b may have a higher laser intensity and/or laser power in the second set of surgical parameters 414b than the laser intensity in the second sets of surgical parameters 414a and 414c.

In some embodiments, the surgical parameters may vary within the clouded lens portion 406 depending on the biomechanical properties of the clouded lens portion 406. For example, a stiffer region within the clouded lens portion 406a may require a higher laser intensity than a region that is not as stiff in the clouded lens portion 406a. As a result, the second set of surgical parameters 414a of the customized treatment plan 410a may include higher laser intensities for the stiffer region and lower laser intensities in the region that is not as stiff for example. In some embodiments, the surgical parameters may vary within the clouded lens portion 406 depending on the geometric properties of the clouded lens portion 406. For example, the clouded portion 406a may have a star-like shape with a wide-body and multiple narrow points extending from the wide-body. As a result, the second set of surgical parameters 414a may have shorter cuts in the points of the star-like shape of the clouded lens portion 406a than the second set of surgical parameters 414a does in the body of the star-like shape of the clouded portion 406a

In some embodiments, the surgical parameters may vary within the unclouded lens portion 404 depending on the biomechanical properties of the unclouded lens portion 404. For example, a more opaque region within the unclouded lens portion 404a may require a higher laser intensity than a region that is not as opaque in the unclouded lens portion 404a. As a result, the first set of surgical parameters 412a of the customized treatment plan 410a may include higher laser intensities for the more opaque region of the unclouded lens portion 404a and lower laser intensities for the less opaque region of the unclouded lens portion 404a. In some embodiments, the surgical parameters may vary within the unclouded lens portion 404 depending on the geometric properties of the unclouded lens portion 404. For example, regions of the unclouded lens portion 404a may be thicker or have more depth than other parts of the unclouded lens portion 404a. As a result, the first set of surgical parameters 412a may include higher laser intensities and/or laser power for the thicker regions of the unclouded lens portion 404a than the other regions of the unclouded lens portion 404a.

By determining the biomechanical properties and/or geometric properties of the optical imaging results 400a-400c, the customized treatment plans 410a-410c generated may provide for more effective and efficient removal of the target tissues 402a-402c imaged. Thus, a more personalized and accurate treatment plan may be implemented for cataract removal rather than a generic strategy.

Modifications, additions, or omissions may be made to FIG. 4 without departing from the scope of the present disclosure. For example, the customized treatment plans 410a-410c may include more or less surgical parameters than those described in the first sets of surgical parameters 412a-412c and/or the second sets of surgical parameters 414a-414c. Furthermore, the surgical parameters included in the customized treatment plans 410a-410c are described as being included in sets for purpose of illustration only and need not be included in sets. As such, either or both of the first set of surgical parameters 412 or the second set of surgical parameters 414 may be omitted. In addition, the principles described may be applied to any applicable optical imaging result and are not limited to OCT imaging, OCE, or light attenuation analysis. Furthermore, FIG. 4 describes the example customized treatment plans 410a-410c as pertaining to removal of cataractous lenses. However, in some embodiments, the customized treatment plans 400a-400c may pertain to other ophthalmologic operations and/or non-ophthalmologic operations.

In addition, in some embodiments, the customized treatment plans 400a-c may be generated based on the optical imaging results 400a-400c (e.g., an OCT image of an eye) utilizing an AI model. For example, the first sets of surgical parameters 412 and/or the second sets of surgical parameters 414 may be generated by an AI model based on the biomechanical properties and/or geometric properties of the target tissues 402a-402c determined from the optical imaging results 400a-400c. The biomechanical properties and/or the geometric properties of the target tissues 402a-402c may also be determined by the AI model.

FIG. 5 illustrates an example first customized treatment plan 510 based on a first optical imaging result 500 and an example second customized treatment plan 530 based on a second optical imaging result 520. The first optical imaging result 500 may be an OCT image of a target tissue 502 including an unclouded lens portion 504 and a clouded lens portion 506. In some embodiments, the second optical imaging result 520 may be another OCT image of the target tissue 502 including the unclouded lens portion 504 and an altered lens portion 508. In some embodiments, the altered portion 508 may be a remaining portion of the clouded lens portion 506 after part of the clouded portion 506 has been removed according to the first customized treatment plan 510. Additionally or alternatively, the altered portion 508 may be a remaining portion of the clouded lens portion 506 that has not been treated yet. In some embodiments, the clouded lens portion 506 may be a cataract and the target tissue 502 may be a cataractous lens.

The first customized treatment plan 510 may be generated based on biomechanical properties and/or geometric properties determined from the first optical imaging result 500. For example, the first customized treatment plan 510 may be generated by the treatment plan generation operation 212 of process 200 described with reference to FIG. 2. The first customized treatment plan 510 may be similar to the example customized treatment plans 410 described with reference to FIG. 4.

In some embodiments, and as illustrated in FIG. 5, the first customized treatment plan 510 may be mapped onto the first optical imaging result 500. The first customized treatment plan 510 may include a first set of surgical parameters 512 corresponding to the unclouded lens portion 504 and a second set of surgical parameters 514 respectively corresponding to the clouded lens portion 506. The first set of surgical parameters 512 may be the same as or similar to the first set of surgical parameters 412 described with reference to FIG. 4 and the second set of surgical parameters 514 may be the same as or similar to the first set of surgical parameters 414 described with reference to FIG. 4. As illustrated in FIG. 5, the first set of surgical parameters 512 and the second set of surgical parameters 514 may include a cutting location (represented by the individual lines) and a laser intensity (represented by the widths of the individual lines).

In some embodiments, the first customized treatment plan 510 may be implemented by emitting a laser beam such as from the laser 102 of FIG. 1 and adjusting the laser beam according to the first customized treatment plan 510 to remove a portion of the target tissue 502. For example, a portion of the clouded lens portion 506 may be removed (e.g., including the formation of microcavitation bubbles in the target tissue 510) according to the second set of surgical parameters 514. As another example, a portion of the clouded lens portion 506 may be fragmented or otherwise broken apart according to the second set of surgical parameters 514.

In some embodiments, a second optical imaging result 520 may be received from, for example, an optical imaging system such as the imaging system 106 of FIG. 1. As illustrated in FIG. 5, the second optical imaging result 520 is an image of the target tissue 502, which illustrates the cataractous lens of an eye. In some embodiments, the second optical imaging result 520 may include the unclouded lens portion 504 and an altered portion 508. As illustrated in FIG. 5, the altered portion 508 is the remaining portion of the clouded lens portion 506 after part of the clouded lens portion 506 has been removed and/or treated. In some embodiments, the altered portion 508 may be a remaining portion of the unclouded lens portion 504 after part of the unclouded lens portion 504 has been removed. In some embodiments, the altered portion 508 may be a portion of the unclouded lens portion 504 and a portion of the clouded lens portion 506.

In some embodiments, the second optical imaging result 520 may be received as the first customized treatment plan 510 is being implemented (e.g., as cataract removal surgery is being performed) and a portion of the target tissue 502 has been removed according to the first customized treatment plan 510. For example, the second optical imaging result 520 may be received after the first customized treatment plan 510 has been partially implemented, and a portion of the clouded lens portion 506 has been removed by a femtosecond laser during FLACS according to the first customized treatment plan 510.

In these and other embodiments, the altered portion 508 may be identified in the second optical imaging result 520, and the second customized treatment plan 530 may be generated to reflect the altered portion 508. For example, the second customized treatment plan 530 may be generated by the treatment plan generation operation 212 of process 200 described with reference to FIG. 2. In some embodiments, the second customized treatment plan 530 may be an update of the first customized treatment plan 510.

In some embodiments, and as illustrated in FIG. 5, the second customized treatment plan 530 may be mapped onto the second optical imaging result 520. In some embodiments, the second customized treatment plan 530 may include an updated first set of surgical parameters 532 and an updated second set of surgical parameters 534. In some embodiments, the updated first set of surgical parameters 532 may correspond to the unclouded lens portion 504 and the updated second set of surgical parameters 534 may correspond to the altered portion 508.

In some embodiments, the updated first set of surgical parameters 532 and the updated second set of surgical parameters 534 may respectively be updates to the first set of surgical parameters 512 and the second set of surgical parameters 514 based on the altered portion 508. For example, the updated second set of surgical parameters 534 may reflect that a portion of the clouded lens portion 506 has been removed in that the higher laser intensity (the thicker lines) included in the second set of surgical parameters 514 is updated to correspond to the altered portion 508 in the updated second set of surgical parameters 534. As a result, the surgical parameters such as, for example, the laser intensity may be adjusted in substantially real-time to reflect the most current state of the target tissue 502 based on the second optical imaging result 520.

In some embodiments, the second customized treatment plan 530 may be generated based on the biomechanical properties and/or the geometric properties determined from the second optical imaging result 520. For example, as portions of the target tissue 502 are removed according to the first customized treatment plan 510, the biomechanical properties and/or the geometric properties of the target tissue 502 may be re-determined from the second optical imaging result 520 in order to ensure that the biomechanical properties and the geometric properties of the target tissue 502 are current. In some embodiments, the second customized treatment plan 530 may be generated based on the new determinations of the biomechanical properties and/or the geometric properties of the target tissue 502.

In some embodiments, a map of the target tissue 502 may be generated in substantially real-time to reflect the altered portion 508 of the target tissue 502. For example, a 2D map (such as the 2D maps 302 of FIG. 3) and/or a 3D map (such as the 3D map 304 of FIG. 3) may be generated to reflect the altered portion 508 of the target tissue 502. In some embodiments, the second customized treatment plan 530 may be based on the map of the target tissue 502.

Modifications, additions, or omissions may be made to FIG. 5 without departing from the scope of the present disclosure. For example, the second customized treatment plan 530 may include more or less surgical parameters than those described in the updated first set of surgical parameters 532, and/or the updated second set of surgical parameters 534. In addition, the principles described may be applied to any applicable optical imaging result and are not limited to OCT imaging, OCE, or light attenuation analysis. Furthermore, FIG. 5 describes the first customized treatment plan 510 and the second customized treatment plan 530 as pertaining to removal of cataractous lenses. However, in some embodiments, the treatment plans may pertain to other ophthalmologic operations and/or non-ophthalmologic operations.

In addition, in some embodiments, the second customized treatment plan 530 may be generated based on the second optical imaging result 520 utilizing an AI model. For example, the updated first set of surgical parameters 532 and/or the updated second set of surgical parameters 534 may be generated by an AI model based on the biomechanical properties and/or geometric properties of the target tissue 502.

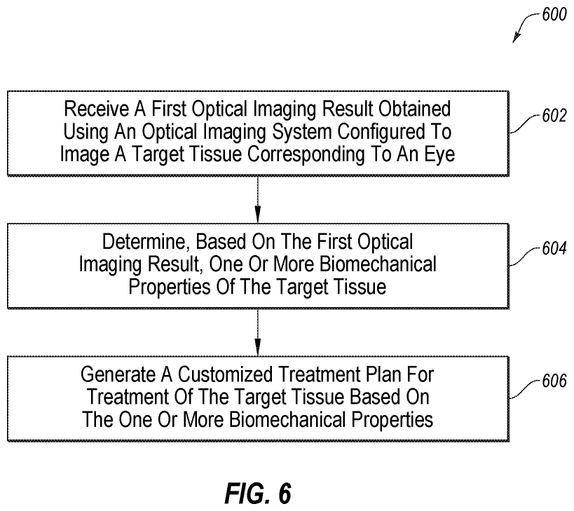

FIG. 6 is a flow diagram illustrating a method 600 of generating a customized treatment plan for a target tissue based on optical images, according to one or more embodiments of the present disclosure. One or more operations of the method 600 may be performed by any suitable system, apparatus, or device such as, for example, the system 100 of FIG. 1, and/or a computing system such as that described with respect to FIG. 7 of the present disclosure. Furthermore, one or more operations of the method 600 may be performed by an AI model. In addition, the method 600 may be performed as part of the process 200 described with respect to FIG. 2.

At block 602, a first optical imaging result may be received. The first optical imaging result may be received from an optical imaging system that may image a target tissue corresponding to an eye. In some embodiments, the target tissue may be a cataractous lens of an eye. In some embodiments, the first optical imaging result may be similar to or the same as the optical imaging result 204 described with reference to FIG. 2, the optical imaging results 400a-400c described with reference to FIG. 4, and/or the first optical imaging result 500 described with reference to FIG. 5. In some embodiments, the optical imaging system may be similar to or the same as the imaging system 106 described with reference FIG. 1 and/or the optical imaging system 202 described with reference to FIG. 2. In some embodiments, the optical imaging system may include an OCT imaging system. In some embodiments, the target tissue may be similar or the same as the target tissue 110 described with reference to FIG. 1, the cataractous lens 314 described with reference to FIG. 3, the target tissues 402a-402c described with reference to FIG. 4, and/or the target tissue 502 described with reference to FIG. 5.

At block 604, one or more biomechanical properties of the target tissue may be determined based on the first optical imaging result. In some embodiments, the one or more biomechanical properties may include at least one of opacity, elasticity, stiffness, density, light scattering intensity, light attenuation, grading, nuclear severity, cortical severity, posterior subcapsular severity, type of target tissue, strain, Poisson's ratio, or transparency. In some embodiments, the biomechanical properties determined may be the same as or similar to the biomechanical properties 208 of FIG. 2 and/or the biomechanical properties described elsewhere in the present application.

In some embodiments, in addition to the biomechanical properties, one or more geometric properties of the target tissue may be determined based on the first optical imaging result. In some embodiments, the geometric properties may include at least one of the size, shape, structure, or dimensions of the target tissue. In some embodiments, the geometric properties determined may be the same as or similar to the geometric properties 210 of FIG. 2 and/or the geometric properties described elsewhere in the present application. While described as both being determined based on the first optical imaging result, it will be appreciated that in some embodiments, one or more biomechanical properties may be based on the first optical imaging result and one or more geometric properties may be based on a second optical imaging result, or one or both of the biomechanical properties and the geometric properties may be based on multiple optical imaging results.

At block 606, a customized treatment plan for treatment of the target tissue may be generated based on the one or more biomechanical properties and/or the one or more geometric properties. In some embodiments, the customized treatment plan may include a three-dimensional plan for removing at least a portion of the target tissue. As indicated in the present disclosure, the removal of at least a portion of the target tissue may include the formation of microcavitation bubbles in the target tissue. In some embodiments, the customized treatment plan may include one or more surgical parameters corresponding to at least a portion of the target tissue. In some of these embodiments, the surgical parameters may include one or more of a laser parameter, a cutting location, a cutting trajectory, a cutting depth, ultrasound energy, or surgical tool selection. In some embodiments, the laser parameter may include laser intensity, laser power, laser duration, and/or laser trajectory.

In some embodiments, a map may be generated of the target tissue in three-dimensional space based on the one or more biomechanical properties and/or the one or more geometric properties. In at least some of these embodiments, the customized treatment plan may be generated based on the map of the target tissue.

In some embodiments, the method 600 may further include receiving a second optical imaging result obtained using the optical imaging system. In some embodiments, an altered portion of the target tissue may be identified based on the second optical imaging result. In some embodiments, a map of the target tissue may be generated in substantially real-time to reflect the altered portion of the target tissue. In some embodiments, the customized treatment plan may be updated based on the map of the target tissue reflecting the altered portion of the target tissue.

In some embodiments, the method 600 may further include emitting a laser beam to perform one or more ophthalmic treatment operations. In these and other embodiments, the laser beam may be adjusted according to the customized treatment plan.

Modifications, additions, or omissions may be made to the method 600 without departing from the scope of the present disclosure. For example, the operations of method 600 may be implemented in differing order in some instances. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

### Example Computing System

FIG. 7 is a block diagram of an example computing system 700 suitable for use in implementing some embodiments of the present disclosure. Computing system 700 may include an interconnect system 702 that directly or indirectly couples the following devices: memory 704, one or more central processing units (CPUs) 706, one or more graphics processing units (GPUs) 708, a communication interface 710, I/O ports 712, input/output components 714, a power supply 716, one or more presentation components 718 (e.g., display(s)), and one or more logic units 720.

Although the various blocks of FIG. 7 are illustrated as connected via the interconnect system 702 with lines, this is not intended to be limiting and is for clarity only. For example, in some embodiments, a presentation component 718, such as a display device, may be considered an I/O component 714 (e.g., if the display is a touch screen). As another example, the CPUs 706 and/or GPUs 708 may include memory (e.g., the memory 704 may be representative of a storage device in addition to the memory of the GPUs 708, the CPUs 706, and/or other components). In other words, the computing system of FIG. 7 is merely illustrative. Distinction is not made between such categories as “workstation,” “server,” “laptop,” “desktop,” “tablet,” “client device,” “mobile device,” “hand-held device,” “game console,” “electronic control unit (ECU),” “virtual reality system,” “augmented reality system,” and/or other device or system types, as all are contemplated within the scope of the computing system of FIG. 7.

The interconnect system 702 may represent one or more links or busses, such as an address bus, a data bus, a control bus, or a combination thereof. The interconnect system 702 may include one or more bus or link types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus or link. In some embodiments, there are direct connections between components. As an example, the CPU 706 may be directly connected to the memory 704. Further, the CPU 706 may be directly connected to the GPU 708. Where there is direct, or point-to-point, connection between components, the interconnect system 702 may include a PCIe link to carry out the connection. In these examples, a PCI bus need not be included in the computing system 700.

The memory 704 may include any of a variety of computer-readable media. The computer-readable media may be any available media that may be accessed by the computing system 700. The computer-readable media may include both volatile and nonvolatile media, and removable and non-removable media. By way of example, and not limitation, the computer-readable media may comprise computer-storage media and communication media.

The computer-storage media may include both volatile and nonvolatile media and/or removable and non-removable media implemented in any method or technology for storage of information such as computer-readable instructions, data structures, program modules, and/or other data types. For example, the memory 704 may store computer-readable instructions (e.g., that represent a program(s) and/or a program element(s), such as an operating system. Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information and that may be accessed by computing system 700. As used herein, computer storage media does not comprise signals per se.

The computer storage media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the computer storage media may include wired media such as a wired network or direct-wired connection, and wireless media such as acoustic, RF, infrared and other wireless media. Combinations of any of the above should also be included within the scope of computer-readable media.

The CPU(s) 706 may be configured to execute at least some of the computer-readable instructions to control one or more components of the computing system 700 to perform one or more of the methods and/or processes described herein. The CPU(s) 706 may each include one or more cores (e.g., one, two, four, eight, twenty-eight, seventy-two, etc.) that are capable of handling a multitude of software threads simultaneously. The CPU(s) 706 may include any type of processor, and may include different types of processors depending on the type of computing system 700 implemented (e.g., processors with fewer cores for mobile devices and processors with more cores for servers). For example, depending on the type of computing system 700, the processor may be an Advanced RISC Machines (ARM) processor implemented using Reduced Instruction Set Computing (RISC) or an x86 processor implemented using Complex Instruction Set Computing (CISC). The computing system 700 may include one or more CPUs 706 in addition to one or more microprocessors or supplementary co-processors, such as math co-processors.

In addition to or alternatively from the CPU(s) 706, the GPU(s) 708 may be configured to execute at least some of the computer-readable instructions to control one or more components of the computing system 700 to perform one or more of the methods and/or processes described herein. One or more of the GPU(s) 708 may be an integrated GPU (e.g., with one or more of the CPU(s) 706 and/or one or more of the GPU(s) 708 may be a discrete GPU. In embodiments, one or more of the GPU(s) 708 may be a coprocessor of one or more of the CPU(s) 706. The GPU(s) 708 may be used by the computing system 700 to render graphics (e.g., 3D graphics) or perform general purpose computations. For example, the GPU(s) 708 may be used for General-Purpose computing on GPUs (GPGPU). The GPU(s) 708 may include hundreds or thousands of cores that are capable of handling hundreds or thousands of software threads simultaneously. The GPU(s) 708 may generate pixel data for output images in response to rendering commands (e.g., rendering commands from the CPU(s) 706 received via a host interface). The GPU(s) 708 may include graphics memory, such as display memory, for storing pixel data or any other suitable data, such as GPGPU data. The display memory may be included as part of the memory 704. The GPU(s) 708 may include two or more GPUs operating in parallel (e.g., via a link). The link may directly connect the GPUs (e.g., using NVLINK) or may connect the GPUs through a switch (e.g., using NVSwitch). When combined together, each GPU 708 may generate pixel data or GPGPU data for different portions of an output or for different outputs (e.g., a first GPU for a first image and a second GPU for a second image). Each GPU may include its own memory, or may share memory with other GPUs.

In addition to or alternatively from the CPU(s) 706 and/or the GPU(s) 708, the logic unit(s) 720 may be configured to execute at least some of the computer-readable instructions to control one or more components of the computing system 700 to perform one or more of the methods and/or processes described herein. In embodiments, the CPU(s) 706, the GPU(s) 708, and/or the logic unit(s) 720 may discretely or jointly perform any combination of the methods, processes and/or portions thereof. One or more of the logic units 720 may be part of and/or integrated in one or more of the CPU(s) 706 and/or the GPU(s) 708 and/or one or more of the logic units 720 may be discrete components or otherwise external to the CPU(s) 706 and/or the GPU(s) 708. In embodiments, one or more of the logic units 720 may be a coprocessor of one or more of the CPU(s) 706 and/or one or more of the GPU(s) 708.

Examples of the logic unit(s) 720 include one or more processing cores and/or components thereof, such as Tensor Cores (TCs), Tensor Processing Units (TPUs), Pixel Visual Cores (PVCs), Vision Processing Units (VPUs), Graphics Processing Clusters (GPCs), Texture Processing Clusters (TPCs), Streaming Multiprocessors (SMs), Tree Traversal Units (TTUs), Artificial Intelligence Accelerators (AIAs), Deep Learning Accelerators (DLAs), Arithmetic-Logic Units (ALUs), Application-Specific Integrated Circuits (ASICs), Floating Point Units (FPUs), I/O elements, peripheral component interconnect (PCI) or peripheral component interconnect express (PCIe) elements, and/or the like.

The communication interface 710 may include one or more receivers, transmitters, and/or transceivers that enable the computing system 700 to communicate with other computing systems via an electronic communication network, including wired and/or wireless communications. The communication interface 710 may include components and functionality to enable communication over any of a number of different networks, such as wireless networks (e.g., Wi-Fi, Z-Wave, Bluetooth, Bluetooth LE, ZigBee, etc.), wired networks (e.g., communicating over Ethernet or InfiniBand), low-power wide-area networks (e.g., LoRaWAN, SigFox, etc.), and/or the Internet.

The I/O ports 712 may enable the computing system 700 to be logically coupled to other devices including the I/O components 714, the presentation component(s) 718, and/or other components, some of which may be built into (e.g., integrated in) the computing system 700. Illustrative I/O components 714 include a microphone, mouse, keyboard, joystick, game pad, game controller, satellite dish, scanner, printer, wireless device, etc. The I/O components 714 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of the computing system 700. The computing system 700 may include depth cameras, such as stereoscopic camera systems, infrared camera systems, RGB camera systems, touchscreen technology, and combinations of these, for gesture detection and recognition. Additionally, the computing system 700 may include accelerometers or gyroscopes (e.g., as part of an inertia measurement unit (IMU)) that enable detection of motion. In some examples, the output of the accelerometers or gyroscopes may be used by the computing system 700 to render immersive augmented reality or virtual reality.

The power supply 716 may include a hard-wired power supply, a battery power supply, or a combination thereof. The power supply 716 may provide power to the computing system 700 to enable the components of the computing system 700 to operate.

The presentation component(s) 718 may include a display (e.g., a monitor, a touch screen, a television screen, a heads-up-display (HUD), other display types, or a combination thereof), speakers, and/or other presentation components. The presentation component(s) 718 may receive data from other components (e.g., the GPU(s) 708, the CPU(s) 706, etc.), and output the data (e.g., as an image, video, sound, etc.).

Modifications, additions, or omissions may be made to FIG. 7 without departing from the scope of the present disclosure. For example, the computing system 700 may include more or fewer elements depending on the implementation. Further, the computing system 700 may be configured to perform any number of operations as compared to those explicitly described.

The disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to codes that perform particular tasks or implement particular abstract data types. The disclosure may be practiced in a variety of system configurations, including hand-held devices, consumer electronics, general-purpose computers, more specialty computing systems, etc. The disclosure may also be practiced in distributed computing environments where tasks are performed by remote-processing devices that are linked through a communications network.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B. Additionally, use of the term “based on” should not be interpreted as “only based on” or “based only on.” Rather, a first element being “based on” a second element includes instances in which the first element is based on the second element but may also be based on one or more additional elements.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

The subject technology of the present disclosure is illustrated, for example, according to various aspects described below. Various examples of aspects of the present disclosure are described as numbered examples (1, 2, 3, etc.) for convenience. These are provided as examples and do not limit the present disclosure. The aspects of the various implementations described herein may be omitted, substituted for aspects of other implementations, or combined with aspects of other implementations unless context dictates otherwise. For example, one or more aspects of example 1 below may be omitted, substituted for one or more aspects of another example (e.g., example 2) or examples, or combined with aspects of another example The following is a non-limiting summary of some example implementations presented herein.

Example 1. A system comprising:
a computing system configured to cause performance of operations, the operations comprising:
   determining, based on a first optical imaging result obtained using an optical imaging system, one or more biomechanical properties of a target tissue corresponding to an eye;
   determining, based on the first optical imaging result, one or more geometric properties of the target tissue; and
   generating a customized treatment plan for treatment of the target tissue based on the one or more biomechanical properties and the one or more geometric properties.

Example 2. The system of Example 1, wherein the operations further comprise:
generating a map of the target tissue in three-dimensional space based on the one or more biomechanical properties and the one or more geometric properties; and
the customized treatment plan is generated based on the map of the target tissue.

Example 3. The system of Example 1 or Example 2 wherein the customized treatment plan includes a three-dimensional plan for removing at least a portion of the target tissue.

Example 4. The system of any of Examples 1-3, wherein the target tissue is a cataractous lens of the eye.

Example 5. The system of any of Examples 1-4, wherein the operations further comprise:
identifying, based on a second optical imaging result obtained using the optical imaging system, an altered portion of the target tissue; and
generating a map of the target tissue in substantially real-time to reflect the altered portion of the target tissue.

Example 6. The system of Example 5, wherein the operations further comprise updating the customized treatment plan based on the map of the target tissue.

Example 7. The system of any of Examples 1-6, wherein an artificial intelligence (AI) model is used for, based on the first optical imaging result, one or more of:
determining the one or more biomechanical properties;
determining the one or more geometric properties; or
generating the customized treatment plan.

Example 7. The system Example 5 or Example 6, wherein an artificial intelligence (AI) model is used for, based on the second optical imaging result, one or more of:
identifying the altered portion of the target tissue; or
updating the customized treatment plan based on the map of the target tissue.

Example 8. The system of any of Examples 1-7, wherein the one or more biomechanical properties of the target tissue include one or more of:
opacity of the target tissue;
elasticity of the target tissue;
viscoelasticity of the target tissue;
stiffness of the target tissue;
density of the target tissue;
light scattering intensity of the target tissue;
light attenuation of target tissue;
grading of the target tissue;
nuclear severity of the target tissue;
cortical severity of the target tissue;
posterior subcapsular severity of the target tissue;
type of target tissue;
strain of the target tissue;
shear modulus of the target tissue;
Poisson's ratio of the target tissue; or
transparency of the target tissue.

Example 9. The system of any of Examples 1-8, wherein the customized treatment plan includes one or more surgical parameters corresponding to at least a portion of the target tissue, the one or more surgical parameters including one or more of:
a laser parameter, a cutting location, a cutting trajectory, a cutting depth, ultrasound energy, or surgical tool selection.

Example 10. The system of Example 9, wherein the laser parameter includes one or more of:
laser intensity;
laser power;
laser duration; or
laser trajectory.

Example 11. The system of any of Examples 1-10, wherein the optical imaging system includes an Optical Coherence Tomography (OCT) imaging system.

Example 12. The system of any of Examples 1-11, further comprising a laser configured to emit a laser beam used to perform one or more ophthalmic treatment operations, the laser beam being adjusted based on the customized treatment plan.

Example 13. The system of any of Examples 1-12, further comprising, the optical imaging system configured to image the target tissue.

Example 14. A method performed by the system of any of Examples 1-13.

What is claimed is:

1. A system comprising:
a computing system configured to cause performance of operations, the operations comprising:
determining, based on a first optical imaging result obtained using an optical imaging system, one or more biomechanical properties of a target tissue corresponding to an eye;
determining, based on the first optical imaging result, one or more geometric properties of the target tissue; and
generating a customized treatment plan for treatment of the target tissue based on the one or more biomechanical properties and the one or more geometric properties.

2. The system of claim 1, wherein:
the operations further comprise generating a map of the target tissue in three-dimensional space based on the one or more biomechanical properties and the one or more geometric properties; and
the customized treatment plan is generated based on the map of the target tissue.

3. The system of claim 1, wherein the customized treatment plan includes a three-dimensional plan for removing at least a portion of the target tissue.

4. The system of claim 1, wherein the target tissue is a cataractous lens of the eye.

5. The system of claim 1, wherein the operations further comprise:
identifying, based on a second optical imaging result obtained using the optical imaging system, an altered portion of the target tissue; and
generating a map of the target tissue in substantially real-time to reflect the altered portion of the target tissue.

6. The system of claim 5, wherein the operations further comprise:
updating the customized treatment plan based on the map of the target tissue.

7. The system of claim 5, wherein an artificial intelligence (AI) model is used for, based on the second optical imaging result, one or more of:
identifying the altered portion of the target tissue; or
updating the customized treatment plan based on the map of the target tissue.

8. The system of claim 1, wherein an artificial intelligence (AI) model is used for, based on the first optical imaging result, one or more of:
determining the one or more biomechanical properties;
determining the one or more geometric properties; or
generating the customized treatment plan.

9. The system of claim 1, wherein the one or more biomechanical properties of the target tissue include one or more of:
opacity of the target tissue;
elasticity of the target tissue;
viscoelasticity of the target tissue;
stiffness of the target tissue;
density of the target tissue;
light scattering intensity of the target tissue;
light attenuation of the target tissue;
grading of the target tissue;
nuclear severity of the target tissue;
cortical severity of the target tissue;
posterior subcapsular severity of the target tissue;
type of target tissue;
strain of the target tissue;
shear modulus of the target tissue;
Poisson's ratio of the target tissue; or
transparency of the target tissue.

10. The system of claim 1, wherein the customized treatment plan includes one or more surgical parameters corresponding to at least a portion of the target tissue, the one or more surgical parameters including one or more of:
a laser parameter, a cutting location, a cutting trajectory, a cutting depth, ultrasound energy, or surgical tool selection.

11. The system of claim 10, wherein the laser parameter includes one or more of:
laser intensity;
laser power;
laser duration; or
laser trajectory.

12. The system of claim 1, wherein the optical imaging system includes an Optical Coherence Tomography (OCT) imaging system.

13. The system of claim 1, further comprising a laser configured to emit a laser beam used to perform one or more ophthalmic treatment operations, the laser beam being adjusted based on the customized treatment plan.

14. A method comprising:
receiving a first optical imaging result obtained using an optical imaging system configured to image a target tissue corresponding to an eye;
determining, based on the first optical imaging result, one or more biomechanical properties of the target tissue; and
generating a customized treatment plan for treatment of the target tissue based on the one or more biomechanical properties.

15. The method of claim 14, further comprising:
generating a map of the target tissue in three-dimensional space based on the one or more biomechanical properties, the customized treatment plan being generated based on the map of the target tissue.

16. The method of claim 14, further comprising:
receiving a second optical imaging result obtained using the optical imaging system;
identifying, based on the second optical imaging result of the optical imaging system, an altered portion of the target tissue; and
generating a map of the target tissue in substantially real-time to reflect the altered portion of the target tissue.

17. The method of claim 14, further comprising:
emitting a laser beam to perform one or more ophthalmic treatment operations; and
adjusting the laser beam based on the customized treatment plan.

18. The method of claim 14, further comprising:
determining, based on the first optical imaging result, one or more geometric properties of the target tissue, and wherein the generating of the customized treatment plan is based on the one or more geometric properties.

19. A system comprising:
an optical imaging system;
a laser configured to emit a laser beam used to perform one or more ophthalmic treatment operations; and
a computing system configured to cause performance of operations, the operations comprising:
determining, based on a first optical imaging result obtained using the optical imaging system, one or more biomechanical properties of a target tissue corresponding to an eye;
determining, based on the first optical imaging result, one or more geometric properties of the target tissue;
generating a customized treatment plan for treatment of the target tissue based on the one or more biomechanical properties and the one or more geometric properties; and
configuring the laser beam based on the customized treatment plan.

20. The system of claim 19, wherein the operations further comprise:
identifying, based on a second optical imaging result obtained using the optical imaging system, an altered portion of the target tissue; and
generating a map of the target tissue in substantially real-time to reflect the altered portion of the target tissue.
